(19) United States
(12) Patent Application Publication
(10) Pub. No.: US 2025/0025498 A1
(43) Pub. Date: Jan. 23, 2025

(54) Methods and Compositions for Xenotransplantation

(71) Applicant: The Trustees of Columbia University in the City of New York, New York, NY (US)

(72) Inventors: Megan Sykes, New York, NY (US); Yong-Guang Yang, New York, NY (US)

(21) Appl. No.: 18/688,190

(22) PCT Filed: Sep. 1, 2022
(86) PCT No.: PCT/US2022/075809

§ 371 Date: Feb. 29, 2024

Related U.S. Application Data
(60) Provisional application No. 63/240,637, filed on Sep. 3, 2021.

(51) Int. Cl. A61K 35/12; C12N 5/071

(52) U.S. Cl. CPC A61K 35/12; C12N 5/0602; A61K 2035/122; C12N 2501/599

(57) ABSTRACT

Provided herein are methods of xenotransplantation, for example, porcine to human transplantation. Also provided herein are extracellular vesicles (“EVs”, e.g., exosomes) and compositions comprising the same. e.g. EVs expressing human CD47. Further provided herein are methods of making EVs and use thereof for xenotransplantation. In some aspects, the methods of xenotransplantation comprise steps of expressing human CD47 on xenografts.

### CROSS REFERENCE

This application claims the benefit of U.S. Provisional Application No. 63/240,637, filed Sep. 3, 2021, the full disclosure of which is hereby incorporated by reference herein in its entirety.

### GOVERNMENT LICENSE RIGHTS

This invention was made with government support under grant number P01 AI045897 awarded by National Institute of Allergy and Infectious Diseases (NIAID), National Institutes of Health (NIH). The government has certain rights in the invention.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14648-004-228_seqlist.xml,” was created on Aug. 30, 2022, and is 32,782 bytes in size.

### INTRODUCTION

Provided herein are methods of xenotransplantation, for example, porcine to human transplantation. Also provided herein are extracellular vesicles (“EVs”, e.g., exosomes) and compositions comprising the same, e.g. EVs expressing human CD47. Further provided herein are methods of making EVs and use thereof for xenotransplantation. In some aspects, the methods of xenotransplantation comprise steps of expressing human CD47 on xenografts. In certain aspects, the method of xenotransplantation is independent of Signal Regulatory Protein a (SIRPa) expression on the target tissue.

### BACKGROUND OF THE INVENTION

The severe shortage of allogeneic donors currently limits the number of organ transplants performed. This supply-demand disparity may be corrected by the use of organs from other species (xenografts). In view of the ethical issues and impracticalities associated with the use of non-human primates, pigs are considered the most suitable donor species for humans. In addition to organ size and physiologic similarities to humans, the ability to rapidly breed and inbreed pigs makes them particularly amenable to genetic modifications that could improve their ability to function as graft donors to humans (Sachs 1994, Path. Biol. 42:217-219; Piedrahita et al., 2004, Am. J. Transplant, 4 Suppl. 6:43-50).

Although transplantation coupled with non-specific immunosuppressive therapy is associated with high early graft tolerance, a major limitation to the success of clinical organ transplantation has been late graft loss, due largely to chronic rejection of the transplant. An average of 4.4 life-years per recipient is saved by a kidney transplant. See Rana et al. JAMA Surg. 2015; 150 (3): 252-259. However, more than 30% of grafts fail within 10 years after living donor kidney. See Department of Health and Human Services: 2017 Annual Data Report: Kidney [retrieved on Mar. 22, 2021]. Retrieved from the Internet: <URL: srtr.transplant.hrsa.gov/annual_reports/2017/Kidney.aspx>.

Immune tolerance is more important for successful clinical xenotransplantation, as the level of life-long immunosuppression required to prevent xenograft rejection can be too toxic to be acceptable. In addition, no markers have been identified to reliably indicate whether or not immunological tolerance has been achieved in patients, resulting in an absence of laboratory parameters upon which to base immunosuppression withdrawal.

Therefore, goals in xenotransplantation include optimizing the durability of mixed chimeric cells originated from the donor animal after they are transplanted into a xenogeneic recipient, as well as maintaining the health and viability of the donor animal.

Mixed chimerism can induce tolerance to the donor at the level of T cells, B cells and natural killer (NK) cells in the recipient. Griesemer A, Yamada K. and Sykes M., 2014, Immunol. Rev. 258:241-258. Sachs D. H., Kawai T. and Sykes M., 2014, Cold Spring Harb. Perspect. Med. 4: a015529. Hematopoiesis is a tightly regulated process involving interactions of cytokines and adhesion molecules in the bone marrow microenvironment with receptors on the hematopoietic cells. Because many of these receptor-ligand interactions are species-specific (e.g., IL-3 and IL-3R) or species-selective (e.g., SCF-cKIT, GM-CSF-GM-CSFR, VLA-5-fibronectin), mixed chimeric cells (e.g., from a pig) will be at a competitive disadvantage compared to endogenous hematopoietic cells (e.g., human cells), resulting in a gradual loss of the transplanted cells. Since durable mixed chimerism can best assure life-long T, B and NK cell tolerance, this loss of chimerism is undesirable.

Introduction of human cytokine receptors and adhesion molecules into a porcine donor animal would help to overcome this competitive disadvantage, assuring lifelong chimerism and tolerance. Griesemer A, Yamada K. and Sykes M., 2014, Immunol. Rev., 258:241-258. Because hematopoiesis is tightly regulated, it may be desirable to insert these genes into their natural locus in the porcine genome so they can function in a physiologic manner under the control of the native regulatory sequence. This may be achieved by disrupting the native porcine gene and replacing it with the human counterpart. However, this approach can have the problem of rendering porcine cells unresponsive or hyporesponsive to species-specific or species-selective porcine cytokines (or adhesion ligands), respectively. Therefore, long-term expression of human transgenes can be deleterious to the health of the donor animal. Dwyer et al., J. Clin. Invest. 2004 May; 113 (10): 1440-6, and Crikis et al., 2010, Am. J. Transplant; 10:242-50.

CD47, also known as integrin-associated protein (IAP), is a ubiquitously expressed 50-kDa cell surface glycoprotein and serves as a ligand for signal regulatory protein (SIRP) a, (also known as CD172a, and SHPS-1) (Brown, 2002, Curr. Opin. Cell. Biol., 14:603-7; Brown and Frazier, 2001, Trends Cell Biol., 111:130-5). CD47 and SIRPa, constitute a cell-cell communication system that plays important roles in a variety of cellular processes including cell migration, adhesion of B cells, and T cell activation (Liu et al., 2002, J. Biol. Chem. 277:10028; Motegi et al., 2003, EMBO 122:2634; Yoshida et al., 2002, J. Immunol. 168:3213; Latour et al., 2001; J. Immunol. 167:2547). In addition, the CD47-SIRPa system is implicated in negative regulation of phagocytosis by macrophages. CD47 on the surface of some cell types (i.e., erythrocytes, platelets or leukocytes) inhibited phagocytosis by macrophages. The role of CD47-SIRPa interaction in the inhibition of phagocytosis has been illustrated by the observation that primary, wild-type mouse macrophages rapidly phagocytose unopsonized red blood cells (RBCs) obtained from CD47-deficient mice but not those from wild-type mice (Oldenborg et al., 2000; Science 288:2051). It has also been reported that through its receptors, SIRPa, CD47 inhibits both Fcγ and complement receptor mediated phagocytosis (Oldenborg et al., J. 2001; Exp. Med. 193:855).

CD47 is ubiquitously expressed and acts as a ligand of signaling regulatory protein (SIRP)α, a critical inhibitory receptor on macrophages and dendritic cells (DCs). Emerging evidence indicate that the CD47-SIRPα signaling pathway plays an important role in regulation of macrophage and DC activation, offering a promising intervention target for immunological disorders. The CD47-SIRPα cell communication system is species-specific (e.g., porcine CD47 does not inhibit phagocytosis of pig bone marrow cells. The lack of cross-reaction between pig CD47 and human SIRPα also contributes to rejection of other types of porcine cells (e.g. hepatocytes) by human macrophages, and stimulates DC activation (see below), and hence elicits anti-pig T cell responses.

CD47-deficient cells are vigorously rejected by macrophages after infusion into syngeneic wild-type (WT) mice, demonstrating that CD47 provides a “don't eat me” signal to macrophages (Oldenborg P A, et al., 2000 Science, 288:2051-4; Wang et al., 2007, Proc Natl Acad Sci USA. 104:13744). Xenotransplantation using pigs as the transplant source has the potential to resolve the severe shortage of human organ donors, a major limiting factor in clinical transplantation (Yang et al., 2007, Nature reviews Immunology. 7:519-31). The strong rejection of xenogeneic cells by macrophages (Abe 2002, The Journal of Immunology 168:621) is largely caused by the lack of functional interaction between donor CD47 and recipient SIRPα (Wang et al., 2007, Blood; 109:836-42, Ide et al., 2007, Proc Natl Acad Sci USA 104:5062-6. Navarro-Alvarez 2014, Cell transplantation, 23:345-54), leading to the development of human CD47 transgenic pigs (Tena et al., 2017, Transplantation 101:316-21; Nomura et al., 2020, Xenotransplantation. 2020; 27: e12549). In addition to macrophages, a sub-population of DCs also express SIRPα (Wang et al., 2007, Proc Natl Acad Sci USA. 104:13744-9, Guilliams et al., 2016, Immunity. 45:669-84). CD47-SIRPα signaling also inhibits DC activation and their ability to prime T cells, and plays an important role in induction of T cell tolerance by donor-specific transfusion (DST) or hepatocyte transplantation (Wang et al., 2007, Proc Natl Acad Sci USA. 104:13744-9, Wang et al., 2014, Cell transplantation 23:355-63. Zhang et al., 2016, Sci Rep. 6:26839). In addition to serving as a “don't eat me”-molecule to inhibiting phagocytosis via interaction with SIRPa, upon ligation to its ligands (e.g., anti-CD47 antibodies, TSP-1, soluble SIRPa) CD47 signaling also induces cell aging or death and suppresses cell proliferation.

### SUMMARY OF THE INVENTION

In one aspect, provided herein is a method of xenotransplantation, the method comprising (a) obtaining an organ from a donor swine; (b) cross-dressing the organ with human CD47; and (c) transplanting the organ into a human recipient. In some embodiments, the cross-dressing step comprises exposing the organ to human CD47 comprising extracellular vesicles (EVs). In some embodiments, the EVs are isolated from human cells. In some embodiments, the cells express recombinant human CD47. In some embodiments, the cells are transgenic cells.

In some embodiments, the cross-dressing is achieved by incubating the organ with EVs expressing human CD47 for 2 hours. In some embodiments, the cross-dressing is achieved by incubating the organ with EVs expressing human CD47 for 6 hours.

In some embodiments, the cross-dressing is achieved by ex vivo perfusing the organ. In some embodiments, the cross-dressing is achieved by in vivo perfusing the donor swine, the human recipient, or a combination thereof.

In some embodiments, the method results in decreased phagocytosis by human macrophages. In some embodiments, the method results in decreased phagocytosis of the cross-dressed organ cells by human macrophages by about 5% to about 25% compared to non-cross-dressed organ cells as measured by FACS analysis of the percentage of CD14-positive cells engulfing cross-dressed cells. In some embodiments, the method results in decreased phagocytosis of the cross-dressed organ cells by human macrophages by about 25% to about 50% compared to a non-cross-dressed organ cells as measured by FACS analysis of the percentage of CD14-positive cells engulfing cross-dressed cells. In some embodiments, the method results in decreased phagocytosis of the cross-dressed organ cells by human macrophages by about 50% to about 75% compared to a non-cross-dressed organ cell as measured by FACS analysis of the percentage of CD14-positive cells engulfing cross-dressed cells. In some embodiments, the method results in decreased phagocytosis of the cross-dressed organ cells by human macrophages by about 75% to about 80% compared to a non-cross-dressed organ cells as measured by FACS analysis of the percentage of CD14-positive cells engulfing cross-dressed cells. In some embodiments, the method results in decreased phagocytosis of the cross-dressed organ cells by human macrophages by about 80% to about 85% compared to a non-cross-dressed organ cells as measured by FACS analysis of the percentage of CD14-positive cells engulfing cross-dressed cells. In some embodiments, the method results in decreased phagocytosis of the cross-dressed organ cells by human macrophages by about 85% to about 90% compared to a non-cross-dressed organ cells as measured by FACS analysis of the percentage of CD14-positive cells engulfing cross-dressed cells. In some embodiments, the method results in decreased phagocytosis of the cross-dressed organ cells by human macrophages by about 90% to about 95% compared to a non-cross-dressed organ cells as measured by FACS analysis of the percentage of CD14-positive cells engulfing cross-dressed cells. In some embodiments, the method results in no detectable phagocytosis of the cross-dressed organ as measured by FACS analysis of the percentage of CD14-positive cells engulfing cross-dressed cells.

In some embodiments, the method results in increased viability of the organ by protection from human macrophages as measured by FACS analysis of the percentage of CD14-positive cells engulfing cross-dressed cells. In some embodiments, the cross-dressed organ evades phagocytosis without induction of apoptosis. In some embodiments, the cross-dressed organ evades phagocytosis and does not exhibit any detectable level of apoptosis. In some embodiments, the cells obtained from the cross-dressed organ exhibit about 5% to about 25% lower levels of apoptosis compared to cells obtained from a non-cross-dressed organ. In some embodiments, the cells obtained from the cross-dressed organ exhibit about 25% to about 50% lower levels of apoptosis compared to cells obtained from a non-cross-dressed organ. In some embodiments, the cells obtained from the cross-dressed organ exhibit about 50% to about 75% lower levels of apoptosis compared to cells obtained from a non-cross-dressed organ. In some embodiments, the cells obtained from the cross-dressed organ exhibit about 75% to about 80% lower levels of apoptosis compared to cells obtained from a non-cross-dressed organ. In some embodiments, the cells obtained from the cross-dressed organ exhibit about 80% to about 85% lower levels of apoptosis compared to cells obtained from a non-cross-dressed organ. In some embodiments, the cells obtained from the cross-dressed organ exhibit about 85% to about 90% lower levels of apoptosis compared to cells obtained from a non-cross-dressed organ. In some embodiments, the cells obtained from the cross-dressed organ exhibit at least 90% lower levels of apoptosis compared to cells obtained from a non-cross-dressed organ. In some embodiments, apoptosis is measured by propidium iodine staining.

In some embodiments, the cross-dressed organ exhibits reduced inflammation, compared to a non-cross-dressed organ. In some embodiments, the cross-dressed organ exhibits about 5% to about 25% reduced inflammation, compared to a non-cross-dressed organ. In some embodiments, the cross-dressed organ exhibits about 25% to about 50% reduced inflammation, compared to a non-cross-dressed organ. In some embodiments, the cross-dressed organ exhibits about 50% to about 75% reduced inflammation, compared to a non-cross-dressed organ. In some embodiments, the cross-dressed organ exhibits about 75% to about 80% reduced inflammation, compared to a non-cross-dressed organ. In some embodiments, the cross-dressed organ exhibits about 80% to about 85% reduced inflammation, compared to a non-cross-dressed organ. In some embodiments, the cross-dressed organ exhibits about 85% to about 90% reduced inflammation, compared to a non-cross-dressed organ. In some embodiments, the cross-dressed organ exhibits at least 90% reduced inflammation, compared to a non-cross-dressed organ.

In some embodiments, the human recipient exhibits reduced systemic inflammation post transplantation, compared to transplantation with a non-cross-dressed organ. In some embodiments, the human recipient exhibits about 5% to about 25% reduced systemic inflammation post transplantation, compared to transplantation with a non-cross-dressed organ. In some embodiments, the human recipient exhibits about 25% to about 50% reduced systemic inflammation post transplantation, compared to transplantation with a non-cross-dressed organ. In some embodiments, the human recipient exhibits about 50% to about 75% reduced systemic inflammation post transplantation, compared to transplantation with a non-cross-dressed organ. In some embodiments, the human recipient exhibits about 75% to about 80% reduced systemic inflammation post transplantation, compared to transplantation with a non-cross-dressed organ. In some embodiments, the human recipient exhibits about 80% to about 85% reduced systemic inflammation post transplantation, compared to transplantation with a non-cross-dressed organ. In some embodiments, the human recipient exhibits about 85% to about 90% reduced systemic inflammation post transplantation, compared to transplantation with a non-cross-dressed organ. In some embodiments, the human recipient exhibits at least 90% reduced systemic inflammation post transplantation, compared to transplantation with a non-cross-dressed organ.

In some embodiments, the organ is a kidney. In some embodiments, the organ is a lung. In some embodiments, the human recipient suffers from renal failure.

In some embodiments, the human recipient requires less immunosuppressive therapy than the standard of care in a comparable clinical setting. In some embodiments, the human recipient requires 10-20% less immunosuppressive therapy than the standard of care in a comparable clinical setting. In some embodiments, the human recipient requires 20-30% less immunosuppressive therapy than the standard of care in a comparable clinical setting. In some embodiments, the human recipient requires 30-40% less immunosuppressive therapy than the standard of care in a comparable clinical setting. In some embodiments, the human recipient requires 40-50% less immunosuppressive therapy than the standard of care in a comparable clinical setting. In some embodiments, the human recipient requires 50-60% less immunosuppressive therapy than the standard of care in a comparable clinical setting. In some embodiments, the human recipient requires 60-70% less immunosuppressive therapy than the standard of care in a comparable clinical setting. In some embodiments, the human recipient requires 70-80% less immunosuppressive therapy than the standard of care in a comparable clinical setting. In some embodiments, the human recipient requires 80-90% less immunosuppressive therapy than the standard of care in a comparable clinical setting. In some embodiments, the human recipient requires at least 90% less immunosuppressive therapy than the standard of care in a comparable clinical setting.

In some embodiments, the method results in a reduction of proteinuria. In some embodiments, the proteinuria is reduced to less than 3 g per 24 hours. In some embodiments, the proteinuria is reduced to 500 mg per 24 hours. In some embodiments, the proteinuria is reduced to 300 mg per 24 hours. In some embodiments, the proteinuria is reduced to 150 mg per 24 hours.

In some embodiments, the proteinuria resolves within two weeks of the transplant. In some embodiments, the proteinuria resolves within one month of the transplant. In some embodiments, the proteinuria resolves within two months of the transplant. In some embodiments, the proteinuria resolves within four months of the transplant.

In some embodiments, the method further includes transplanting bone marrow tissue into the recipient. In some embodiments, the bone marrow is taken from the same swine as the kidney. In some embodiments, the bone marrow is taken from a different swine than the kidney. In some embodiments, the bone marrow is cross-dressed with human CD47 by exposure to EVs.

In some embodiments, the organ does not express human SIRPα.

### BRIEF DESCRIPTION OF THE DRAWINGS

FIG. 1A-FIG. 1C: Cross-dressing of pig LCL (FIG. 1A) by transgenic hCD47 (FIG. 1B) after co-culture with hCD47-Tg LCL cells (FIG. 1C).

FIG. 2: Cross-dressing of pig LCL and by transgenic hCD47 after co-culture with hCD47-Tg LCL cells.

FIG. 3A and FIG. 3B: Cross-dressing of human Jurkat cells (FIG. 3A) by transgenic hCD47 after co-culture with hCD47-Tg LCL cells (FIG. 3B).

FIG. 4A-FIG. 4C: Cross-dressing of hCD47KO Jurkat cells (FIG. 4A) by native hCD47 (FIG. 4B) after co-culture with WT Jurkat cells (FIG. 4C).

FIG. 5A and FIG. 5B: Cross-dressing of pig LCL (FIG. 5A) by native hCD47 after co-culture with WT Jurkat cells (FIG. 5B).

FIG. 6: CD47 expression on WT Jurkat cells, pig LCL/CD47^{p/h} cells, CD47KO Jurkat cells, CD47KO cells mixed with WT Jurkat cells (mixed at the time of staining), CD47KO Jurkat cells cocultured (24 h) with WT Jurkat or pig hCD47-Tg LCL cells, pig LCL cells, and LCL cells cocultured (24 h) with WT Jurkat cells. The numbers in the figure indicate mean fluorescent intensity (MFI) of CD47 staining on gated CD47KO Jurkat cells and pig LCL cells.

FIG. 7A-7D: Measurement of CD47 cross-dressing of CD47KO Jurkat cells (FIG. 7A) by extracellular vesicles (FIG. 7C) or exosomes (FIG. 7D) from WT Jurkat cells (FIG. 7B) after 2 hours.

FIG. 8A-8D: Measurement of CD47 cross-dressing of CD47KO Jurkat cells (FIG. 8A) by extracellular vesicles (FIG. 8C) or exosomes (FIG. 8D) from WT Jurkat cells (FIG. 8B) after 6 hours.

FIG. 9A-9D: Measurement of CD47 cross-dressing of pig LCL cells (FIG. 9A) by extracellular vesicles (FIG. 9C) or exosomes (FIG. 9D) from WT Jurkat cells after 2 hours.

FIG. 10A-10D: Measurement of CD47 cross-dressing of pig LCL cells (FIG. 10A) by extracellular vesicles (FIG. 10C) or exosomes (FIG. 10D) from WT Jurkat cells after 6 hours.

### DETAILED DESCRIPTION OF THE INVENTION

Provided herein are CD47-carrying extracellular vesicles (“EVs”, e.g., exosomes) and compositions comprising the same. Such CD47-carrying EVs can be used to cross-dress tissues and allow such tissues to evade phagocytic elimination by macrophages and other phagocytes. Such methods and compositions can be used in xenotransplantation. Methods of making EVs are described in section 6.1. Compositions comprising the resulting EVs are described in section 6.2. Methods of using EVs to cross-dress tissues are described in Section 6.3. Uses of such tissues in xenotransplantation are described in section 6.4.

As used herein, the terms “about” or “approximately” mean within plus or minus 10% of a given value or range. In instances where integers are required or expected, and instances of percentages, it is understood that the scope of this term includes rounding up to the next integer and rounding down to the next integer. For clarity, use herein of phrases such as “about X,” and “at least about X,” are understood to encompass and particularly recite “X.”

As used herein, the term “extracellular vesicle (EV)” generally refers to lipid membrane-enclosed vesicles secreted by a cell into the extracellular space, and includes, but is not limited to, exosomes and/or microvesicles.

As used herein, the term “exosome” generally refers to a subset of EVs that are typically smaller in size (e.g., 30-150 nm in diameter) relative to other EVs, such as microvesicles.

As used herein, the term “cross-dressing” generally refers to the expression of a transgenic protein (e.g., CD47) in a cell that is induced by, for example, incubating the cell with cells or EVs expressing said protein.

### 8.1 Methods of Making EVs

### 8.1.1. EVs

Extracellular vesicles (EVs) are lipid bilayer-enclosed membranes released by cells into the extracellular environment. Examples of EVs include exosomes, microvesicles (MVs) and apoptotic bodies. See, e.g., Carnino et al. Respiratory Research (2019) 20:240. In certain embodiments, the EVs comprise exosomes. In some embodiments, the EVs consist of exosomes. In certain embodiments, the EVs comprise MVs. In some embodiments, the EVs consist of MVs.

In particular embodiments, the EVs are about 20 nm to about 2,000 nm. In some embodiments, the EVs are about 20 nm to about 1,500 nm. In some embodiments, the EVs are about 20 nm to about 1,000 nm. In some embodiments, the EVs are about 20 nm to about 500 nm. In some embodiments, the EVs are about 20 nm to about 250 nm. In some embodiments, the EVs are about 20 nm to about 200 nm. In some embodiments, the EVs are about 20 nm to about 150 nm. In some embodiments, the EVs are about 50 nm to about 150 nm. In some embodiments, the EVs are about 50 nm to about 1,500 nm. In some embodiments, the EVs are about 50 nm to about 1,000 nm. In some embodiments, the EVs are about 50 nm to about 500 nm.

Exosomes are one exemplary type of EV suitable for use in the present disclosure. Various markers for characterizing exosomes are known in the art, and include but are not limited to, Alix, Tsg101, tetraspanins (e.g., CD63, CD81, CD82, CD53, and CD37), and flotillin. MVs are another exemplary type of EV suitable for use in the present disclosure, and common protein markers used to define these vesicles include, but are not limited to, selectins, integrins and the CD40 ligand.

### 8.1.2. Sources of EVs

Provided herein are EVs comprising CD47, e.g., human CD47. In some embodiments, the CD47 comprised by the EVs is native to the cell releasing the EV. In other examples, the CD47 is not native to the cell releasing the EV (e.g., transgenic CD47). In a preferred embodiment, the CD47 is transgenic human CD47.

Many cell types release EVs and EVs may carry various types of cargo such as nucleic acids, proteins and lipids which area released by the host cell. The EVs provided herein may be released by cell lines in culture or by primary cells in culture. In exemplary embodiments, the EVs are released from human cells, e.g., human primary cells in culture. In specific embodiments, the EVs provided herein are released from human cells expressing transgenic CD47. In other specific embodiments, the EVs provided herein are released from a human cancer cell, for example from a human cancer cell overexpressing CD47 (e.g., Jurkat leukemia cells). The EVs provided herein may be released from cells that naturally express human CD47, or from cells that have been modified to recombinantly express human CD47, e.g. cells modified as described in section 6.1.3 below. In certain embodiments, the EVs provided herein are released from cells modified to over express human CD47, e.g. cells modified as described in section 6.1.3 below. In certain embodiments, the EVs provided herein are released from cells modified to inducibly express human CD47, e.g. cells modified as described in section 6.1.3 below. In specific embodiments, EVs provided herein are isolated from biological fluids, e.g. from blood.

In some embodiments, cells from which EVs provided herein are released may be treated with agents that enhance EV release. Agents which enhance the release of EVs from cells are well-known in the art, see, e.g., Deng et al., Theranostics 2021, 11 (9): 4351-4362; Wang et al. Cells 2020, 9 (3): 660; and Nakamura et al., Molecular Therapy 28 (10): 2203-2219 October 2020. In a specific embodiment, the agent which enhances EV release is ultrasound, adiponectin, norepinephrine, forskolin, fenoterol, Methyldopamine or mephenesin.

### 8.1.3. Methods of Making Transgenic Cell Lines Releasing EVs

Also provided herein are transgenic cells (e.g., primary cells or cell line cells) expressing CD47, which release CD47-carrying EV. Amino acid sequences of human CD47 can be found under the following NCBI Reference Sequence (RefSeq) accession numbers: NP_001768; NP_001369235.1; NP_942088; and XP_005247966.1. Nucleic acid sequences encoding human CD47 can be found under the following NCBI RefSeq accession numbers: NM_001777; NM_198793; XM_005247909.2 and NM_001382306.1. Any known splice variant of CD47 may be used to make a transgenic cell line provided herein. Non-limiting examples of amino acid and nucleotide sequences of human CD47 are provided in Table 1.

Provided herein are vectors (e.g., expression vectors) comprising polynucleotides comprising nucleotide sequences encoding CD47, e.g., human CD47. Vectors may include viral vector (e.g., an adeno-associated virus (AAV), self-complimentary adeno-associated virus (scAAV), adenovirus, retrovirus, lentivirus (e.g., Simian immunodeficiency virus, human immunodeficiency virus, or modified human immunodeficiency virus), Newcastle disease virus (NDV), herpes virus (e.g., herpes simplex virus), alphavirus, vaccina virus, etc.), a plasmid, or other vector (e.g., non-viral vectors, such as lipoplexes, liposomes, polymerosomes, or nanoparticles).

### 8.1.3.1 Methods of Making Transgenic Cell Lines

Transgenic cells (including primary or cell line cells) may be produced using any method known in the art or provided herein.

A transgenic cell line provided herein may be engineered to express CD47 (e.g., human CD47) using homologous recombination (HR) between a cellular DNA and an exogenous DNA (e.g., a DNA construct, a vector, etc.) introduced into the cell. Alternatively, in some embodiments provided herein, the human CD47 transgene, together with all of its necessary regulatory sequence, is introduced into the cell line, for example, as a human artificial chromosome.

The sequence-specific insertion (or knock-in) of human CD47 transgene into the genome of the cell line may also be achieved by a sequence-specific endonuclease coupled with homologous recombination (HR) of the targeted chromosomal locus with the construct containing the transgene of human CD47. Meyer et al., 2010, Proc. Natl. Acad. Sci. USA 107, 15022-15026. Cui et al., 2010, Nat. Biotechnol. 29:64-67. Moehle et al., 2007, Proc Natl Acad Sci USA 104:3055-3060.

Another example of sequence-specific endonucleases includes RNA-guided DNA nucleases, e.g., the CRISPR/Cas system. The Cas9/CRISPR (Clustered Regularly-Interspaced Short Palindromic Repeats) system exploits RNA-guided DNA-binding and sequence-specific cleavage of target DNA. A guide RNA (gRNA) (e.g., containing 20 nucleotides) are complementary to a target genomic DNA sequence upstream of a genomic PAM (protospacer adjacent motifs) site (NNG) and a constant RNA scaffold region. The Cas (CRISPR-associated) protein binds to the gRNA and the target DNA to which the gRNA binds and introduces a double-strand break in a defined location upstream of the PAM site. Geurts et al., 2009, Science 325:433; Mashimo et al., 2010, PLOS ONE 5, e8870; Carbery et al., 2010, Genetics 186:451-459; Tesson et al., 2011, Nat. Biotech. 29:695-696. Wiedenheft et al. Nature 2012, 482:331-338; Jinek et al. Science, 2012, 337:816-821; Mali et al., 2013, Science 339:823-826; Cong et al. 2012, Science 339:819-823.

The sequence-specific endonuclease of the methods and compositions described herein can be engineered, chimeric, or isolated from an organism. Endonucleases can be engineered to recognize a specific DNA sequence, by, e.g., mutagenesis. Seligman et al. (2002) Nucleic Acids Research 30:3870-3879. Combinatorial assembly is a method where protein subunits form different enzymes can be associated or fused. Amould et al. (2006) Journal of Molecular Biology 355:443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

In some embodiments, a transgenic cell line provided herein inducibly expressed human CD47. Numerous inducible promoters and gene expression systems are known in the art. For example, a promoter may be induced by a chemical, e.g., by tetracyclin, tamoxifen, or cumate. Gene expression can also be controlled by protein-protein interactions (e.g., the interaction between FKBP12 and mTOR, which is controlled by rapamycin). See, e.g., Kallunki et al. (2019), Cells 8:796.

In one embodiment, a sequence-specific recombination system may be used to achieve the conditional knockout of the target gene. The recombinase is an enzyme that recognizes specific polynucleotide sequences (recombinase recognition sites) that flank an intervening polynucleotide and catalyzes a reciprocal strand exchange, resulting in inversion or excision of the intervening polynucleotide. Araki et al., 1995, Proc. Natl. Acad. Sci. USA 92:160-164.

In one embodiment, for a conditional knockout of a target gene in cell, the Cre-loxP system may be used. This involves targeted integration (knock-in) of loxP sites via homologous recombination (HR) and the expression of inducible Cre recombinase.

In another embodiment, conditional expression of the transgene (which encodes, e.g., a recombinase, or human CD47 transgene) can be achieved by using regulatory sequence that can be induced or inactivated by exogenous stimuli. For example, the sequence-specific recombination system of the conditional knock-out allele can be regulated, by, e.g., having the activity of the recombinase to be inducible by a chemical (drug). The chemical may activate the transcription of the Cre recombinase gene, or activates transport of the Cre recombinase protein to the nucleus. Alternatively, the recombinase can be activated by the absence of an administered drug rather than by its presence. Non-limiting examples of the chemicals regulating the inducible system (thus, e.g., inducing conditional knockouts) include tetracycline, tamoxifen, RU-486, doxycycline, and the like. Nagy A (February 2000), Cre recombinase: the universal reagent for genome tailoring, Genesis, 26 (2): 99-109. See, for example, the conditional knock-out and knock-in construct described in U.S. patent application Ser. No. 15/558,789.

### 8.1.3.2 Extrachromosomal Expression of EVs

In certain embodiments, provided herein are methods of producing EVs wherein the CD47 is expressed from extrachromosomal DNA. Extrachromosomal DNA is DNA that does not integrate into the host chromosomal DNA. Non-limiting examples of extrachromosomal DNA include plasmids and circular extrachromosomal DNA. In eukaryotic cells, extrachromosomal DNA may be found inside the nucleus or outside the nucleus. For example, a host cell may be transfected with a vector encoding human CD47 (e.g., a vector such as described in section 6.1.3 above) and the human CD47 protein is expressed from the vector without integrating into the host DNA.

### 8.1.3.3 Isolation of EVs from Cells

EVs may be isolated from cells (e.g., transgenic cells expressing CD47) using any method known in the art or described herein. See, e.g., Carnino et al. Respiratory Research (2019) 20:240.

For example, EVs may be isolated by differential centrifugation of cell culture supernatant. In an exemplary protocol, the cell supernatant is centrifuged at 2,000 g (3,000 rpm) for 20 min to remove cell debris and dead cells. Then EVs are purified by centrifugation at 16,500 g (9,800 rpm) for 45 min. Exosomes may be obtained by a similar protocol, wherein the cell supernatant is centrifuged at 2,000 g (3,000 rpm) for 20 min to remove cell debris and dead cells and exosomes are then isolated by centrifugation at 100,000 g (26,450 rpm) for approximately 2 h to 16 h.

EVs, including exosomes, may also be purified using gradient density centrifugation. In this method, EVs are separated based on their buoyant density in solutions of either sucrose, iohexol, or iodixanol. Other examples of methods used to isolate EVs, such as exosomes, include precipitation with organic solvents (e.g., polyethylene glycol, sodium acetate or protamine), immunoprecipitation, separation using antibody-coated magnetic beads (e.g., anti-CD63 coated magnetic beads), microfluidic devices, and ultrafiltration. See, e.g., Carnino et al. Respiratory Research (2019) 20:240 and Momen-Heravi et al. Biol. Chem. 2013; 394 (10): 1253-1262 for exemplary protocols. Further exemplary methods are isolation using heparin-conjugated agarose beads (see, e.g., Balaj et al. (2015) Sci Rep 5, 10266) and purification using Tim4-affinity purification (see, e.g., Nakai et al. (2016) Sci Rep 6, 33935).

Furthermore, commercial kits for the isolation of EVs are available and may be used to isolate the EVs provided herein. Non-limiting examples include the exoEasy Kit (Qiagen), ExoQuick® kits (Systems Bioscience), Total Exosome Isolation Reagent (ThermoFisher Scientific) and the EasySep™ Human Pan-Extracellular Vesicle Positive Selection Kit (Stem Cell Technologies).

In certain embodiments, the EVs provided herein are isolated or purified. EVs provided herein may be purified using any method known in the art or provided herein. As used herein, an “isolated” or “purified” EV is substantially free of cellular material, microparticles or other contaminants (e.g., organelles, lipids, cholesterol) from the cell or tissue source from which the EV is derived. In specific embodiments, the EVs provided herein are of about 50%, 55%, 60%, 65%, 70%, 75%, 80%, 85%, 90%, 95% 97%, 98%, 99% purity. In a specific embodiment, the EVs provided herein are of more than 99% purity. Purity may be determined, for example by measuring particle size using dynamic light scattering or single particle tracking analysis, or by techniques such as flow cytometry, ELISA, or electron microscopy. See, e.g., Balaj et al. (2015) Sci Rep 5, 10266, Nakai et al. (2016) Sci Rep 6, 33935 and Carnino et al. (2019) Respiratory Research 20:240.

### 8.1.3.4 Assays for Detecting mRNA Levels

Several methods of detecting or quantifying mRNA levels are known in the art. Exemplary methods include, but are not limited to, northern blots, ribonuclease protection assays, PCR-based methods (e.g., quantitative PCR), RNA sequencing, Fluidigm® analysis, and the like. The mRNA sequence of a human CD47 can be used to prepare a probe that is at least partially complementary to the mRNA sequence. The probe can then be used to detect the mRNA in a sample, using any suitable assay, such as PCR-based methods, northern blotting, a dipstick assay, TaqMan™ assays and the like.

In other embodiments, a nucleic acid assay for testing for human CD47 expression in a biological sample can be prepared. An assay typically contains a solid support and at least one nucleic acid contacting the support, where the nucleic acid corresponds to at least a portion of the mRNA. The assay can also have a means for detecting the altered expression of the mRNA in the sample. The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

A typical mRNA assay method can contain the steps of: (1) obtaining surface-bound subject probes; (2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Other methods, such as PCR-based methods, can also be used to detect the expression of human CD47. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). In some embodiments, qRT-PCR-based assays can be useful to measure mRNA levels during cell-based assays. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

### 8.1.3.5 Assays for Detecting Polypeptide or Protein Levels

Several protein detection and quantification methods can be used to measure the level of human CD47. Any suitable protein quantification method can be used. In some embodiments, antibody-based methods are used. Exemplary methods that can be used include, but are not limited to, immunoblotting (Western blot), ELISA, immunohistochemistry, immunofluorescence, flow cytometry, cytometry bead array, mass spectroscopy, and the like. Several types of ELISA are commonly used, including direct ELISA, indirect ELISA, and sandwich ELISA.

### 8.2 EV Compositions

Provided herein are compositions comprising the EVs described herein, e.g., CD47-carrying EVs, such as CD47-carrying exosomes (“EV compositions”). Purified EVs may be cryopreserved, e.g. by freezing EVs in the presence of a cryoprotectant, lyophilized or spray-dried. EVs may be stabilized by using hydrophilic polymers (e.g., polyethylene glycol) or scaffolds (e.g., scaffolds comprising components of the extracellular matrix to which the EVs bind in vivo). See, e.g., Kusuma et al. (2018) Front. Pharmacol., 9:1199.

The EV compositions provided herein may vary in the CD47 content. In certain embodiments, the EV comprises CD47 mRNA. Levels of human CD47 mRNA may be determined by any suitable method known in the art, e.g. a method described in Section 6.1.3.4. In certain embodiments, the EV comprises a CD47 polypeptide or protein. Levels of human CD47 protein may be determined using any suitable method know in the art, e.g., a method described in Section 6.1.3.5.

Thus, for example, at least 50%, at least 60%, at least 70%, at least 80%, at least 90% or at least 95% of the EVs present in a unit of EV composition express human CD47. Similarly, human CD47 may account for at least 50%, at least 60%, at least 70%, at least 80%, at least 90% or at least 95% of the total membrane-associated protein in an EV composition.

The EV compositions provided herein can further include a suitable carrier, e.g., a pharmaceutically acceptable carrier. Generally, a “pharmaceutically acceptable” carrier is a material that is not biologically or otherwise undesirable, i.e., the material can be administered to a subject without causing any undesirable biological effects such as toxicity. Exemplary pharmaceutically acceptable carriers include, but are not limited to, aqueous solvents (e.g., water; balanced salt solutions, such as Phosphate Buffered Saline (PBS), Hanks' balanced salt solution (HSB), Earl's balanced salt solution (EBSS); and cell culture media), as well as non-aqueous solvents (e.g., fats, oils, polyol (for example, glycerol, propylene glycol, and liquid polyethylene glycol, and the like), vegetable oil, and injectable organic esters, such as ethyloleate). The carrier includes liquid, semi-solid, e.g., pastes, or solid carriers. In addition, if desired, the compositions may contain minor amounts of auxiliary substances, such as wetting or emulsifying agents, stabilizing agents, or pH buffering agents. The pH and exact concentration of the various components in a pharmaceutical composition are adjusted according to well-known parameters. Accordingly, the EVs of the invention can be formulated for administration in a pharmaceutically acceptable carrier in accordance with known techniques. See, e.g., Remington, The Science And Practice of Pharmacy (22nd Ed. 2012).

In some embodiments, the composition can contain from 0.01% to 99% weight by volume of the EVs. In some embodiments, the composition can contain from 0.05% to 99% weight by volume of the EVs. In some embodiments, the composition can contain from 0.1% to 99% weight by volume of the EVs. In some embodiments, the composition can contain from 0.5% to 99% weight by volume of the EVs. In some embodiments, the composition can contain from 1% to 99% weight by volume of the EVs. In some embodiments, the composition can contain from 5% to 99% weight by volume of the EVs. In some embodiments, the composition can contain from 10% to 99% weight by volume of the EVs. In some embodiments, the composition can contain from 15% to 99% weight by volume of the EVs. In some embodiments, the composition can contain from 20% to 99% weight by volume of the EVs. In some embodiments, the composition can contain from 25% to 99% weight by volume of the EVs. In some embodiments, the composition can contain from 30% to 99% weight by volume of the EVs. In some embodiments, the composition can contain from 35% to 99% weight by volume of the EVs. In some embodiments, the composition can contain from 40% to 99% weight by volume of the EVs. In some embodiments, the composition can contain from 50% to 99% weight by volume of the EVs. In some embodiments, the composition can contain from 55% to 99% weight by volume of the EVs. In some embodiments, the composition can contain from 60% to 99% weight by volume of the EVs. In some embodiments, the composition can contain from 70% to 99% weight by volume of the EVs. In some embodiments, the composition can contain from 75% to 99% weight by volume of the EVs. In some embodiments, the composition can contain from 80% to 99% weight by volume of the EVs. In some embodiments, the composition can contain from 85% to 99% weight by volume of the EVs. In some embodiments, the composition can contain from 90% to 99% weight by volume of the EVs. In some embodiments, the composition can contain from 95% to 99% weight by volume of the EVs. In some embodiments, the composition can comprise 100% EVs, such as lyophilized EVs.

The amount of EVs included in the compositions provided herein can be readily determined by one skilled in the art. In some embodiments, the amount of EVs is an amount sufficient to cross-dress the xenograft. In specific embodiments, the amount of EVs is an amount sufficient to cross-dress the xenograft and reduce phagocytosis of the organ cells. In some embodiments, the amount of EVs is an amount sufficient to cross-dress the xenograft and reduce systemic inflammation in the recipient post-transplantation.

In certain embodiments, the amount of EVs is a quantified amount. Various methods are known in the art for quantification of EVs, including MVs and exosomes. For example, non-limiting exemplary methods for quantifying EVs include electron microscopy (EM), surface plasmon resonance (SPR), flow cytometry, tunable resistive pulse sensing (TRPS), nanosight nanoparticle Tracking Analysis, protein based methods, and enzyme-linked immunosorbent assay.

In some embodiments, the composition provided herein includes about 1.0×10⁶ to about 1.0×10¹⁵ EVs. In some embodiments, the composition provided herein includes about 1.0×10⁷ to about 1.0×10¹⁴ EVs. In some embodiments, the composition provided herein includes about 1.0×10⁸ to about 1.0×10¹³ EVs. In some embodiments, the composition provided herein includes about 1.0×10⁹ to about 1.0 x10¹² EVs. In some embodiments, the composition provided herein includes about 1.0 x10¹⁰ to about 1.0×10¹¹ EVs. In some embodiments, the composition provided herein includes about 1.0 x10⁶ to about 1.0 x10¹⁰ EVs. In some embodiments, the composition provided herein includes about 1.0×10⁶ to about 1.0×10⁸ EVs. In some embodiments, the composition provided herein includes about 1.0×10⁸ to about 1.0 x10¹⁵ EVs. In some embodiments, the composition provided herein includes about 1.0×10⁸ to about 1.0×10¹² EVs. In some embodiments, the composition provided herein includes about 1.0 x10¹⁰ to about 1.0×10¹⁵ EVs. In some embodiments, the composition provided herein includes about 1.0×10¹² to about 1.0×10¹⁵ EVs.

In some embodiments, the composition provided herein includes about 1.0×10⁶ EVs. In some embodiments, the composition provided herein includes about 1.0×10⁷ EVs. In some embodiments, the composition provided herein includes about 1.0×10⁸ EVs. In some embodiments, the composition provided herein includes about 1.0×10⁹ EVs. In some embodiments, the composition provided herein includes about 1.0×10¹⁰ EVs. In some embodiments, the composition provided herein includes about 1.0×10¹¹ EVs. In some embodiments, the composition provided herein includes about 1.0×10¹² EVs. In some embodiments, the composition provided herein includes about 1.0×10¹³ EVs. In some embodiments, the composition provided herein includes about 1.0 ×10¹⁴ EVs. In some embodiments, the composition provided herein includes about 1.0×10¹⁵ EVs.

In some embodiments, the composition provided herein includes about 1.0 micrograms (μg) to about 100 grams (g) EV protein. In some embodiments, the composition provided herein includes about 5.0 μg to about 50 g EV protein. In some embodiments, the composition provided herein includes about 10.0 μg to about 10 g EV protein. In some embodiments, the composition provided herein includes about 50.0 μg to about 5 g EV protein. In some embodiments, the composition provided herein includes about 100.0 μg to about 1 g EV protein. In some embodiments, the composition provided herein includes about 1.0 μg EV protein. In some embodiments, the composition provided herein includes about 5.0 μg EV protein. In some embodiments, the composition provided herein includes about 10.0 μg EV protein. In some embodiments, the composition provided herein includes about 25.0 μg EV protein. In some embodiments, the composition provided herein includes about 50.0 μg EV protein. In some embodiments, the composition provided herein includes about 100.0 μg EV protein. In some embodiments, the composition provided herein includes about 250.0 μg EV protein. In some embodiments, the composition provided herein includes about 500.0 μg EV protein. In some embodiments, the composition provided herein includes about 1.0 mg EV protein. In some embodiments, the composition provided herein includes about 5.0 mg EV protein. In some embodiments, the composition provided herein includes about 10.0 mg EV protein. In some embodiments, the composition provided herein includes about 25.0 mg EV protein. In some embodiments, the composition provided herein includes about 50.0 mg EV protein. In some embodiments, the composition provided herein includes about 100.0 mg EV protein. In some embodiments, the composition provided herein includes about 250.0 mg EV protein. In some embodiments, the composition provided herein includes about 500.0 mg EV protein. In some embodiments, the composition provided herein includes about 1.0 g EV protein. In some embodiments, the composition provided herein includes about 5.0 g EV protein. In some embodiments, the composition provided herein includes about 10.0 g EV protein. In some embodiments, the composition provided herein includes about 25.0 g EV protein. In some embodiments, the composition provided herein includes about 50.0 g EV protein. In some embodiments, the composition provided herein includes about 100.0 g EV protein. In some embodiments, the composition provided herein includes about 250.0 g EV protein. In some embodiments, the composition provided herein includes about 500.0 g EV protein.

In some embodiments, the amount of EVs included in the composition is relative to the amount of cells from which the EVs are generated. For example, in some embodiments, the amount of EVs is an amount collected from about 1.0×10⁶ to about 1.0×10¹⁰ cells cultured for greater than 48 h. In some embodiments, the amount of EVs is an amount collected from about 1.0×10⁶ to about 1.0×10¹⁰ cells cultured for about 48 h. In some embodiments, the amount of EVs is an amount collected from about 1.0×10⁶ to about 1.0×10¹⁰ cells cultured for about 24 h. In some embodiments, the amount of EVs is an amount collected from about 1.0×10⁶ to about 1.0×10¹⁰ cells cultured for about 16 h. In some embodiments, the amount of EVs is an amount collected from about 1.0×10⁶ to about 1.0 x10¹⁰ cells cultured for about 12 h. In some embodiments, the amount of EVs is an amount collected from about 1.0×10⁶ to about 1.0 x10¹⁰ cells cultured for less than 12 h.

In some embodiments, the amount of EVs is an amount collected from about 1.0×10⁸ to about 1.0 x10¹⁰ cells cultured for greater than 48 h. In some embodiments, the amount of EVs is an amount collected from about 1.0×10⁸ to about 1.0 x10¹⁰ cells cultured for about 24 h. In some embodiments, the amount of EVs is an amount collected from about 1.0×10⁸ to about 1.0×10¹⁰ cells cultured for about 16 h. In some embodiments, the amount of EVs is an amount collected from 1.0×10⁶ to about 1.0×10¹⁰ cells cultured for about 12 h. In some embodiments, the amount of EVs is an amount collected from 1.0×10⁸ to about 1.0×10¹⁰ cells cultured for less than 12 h.

In some embodiments, the amount of EVs is an amount collected from about 1.0×10⁶ to about 1.0×10⁸ cells cultured for greater than 48 h. In some embodiments, the amount of EVs is an amount collected from 1.0×10⁶ to about 1.0×10⁸ cells cultured for about 24 h. In some embodiments, the amount of EVs is an amount collected from 1.0×10⁶ to about 1.0×10⁸ cells cultured for about 16 h. In some embodiments, the amount of EVs is an amount collected from 1.0×10⁶ to about 1.0×10⁸ cells cultured for about 12 h. In some embodiments, the amount of EVs is an amount collected from 1.0×10⁶ to about 1.0×10⁸ cells cultured for less than 12 h.

In some embodiments, the amount of EVs is an amount collected from about 1.0×10⁵ cells. In some embodiments, the amount of EVs is an amount collected from about 1.0×10⁶ cells. In some embodiments, the amount of EVs is an amount collected from about 1.0 x10⁷ cells. In some embodiments, the amount of EVs is an amount collected from about 1.0×10⁸ cells. In some embodiments, the amount of EVs is an amount collected from about 1.0 x10⁹ cells. In some embodiments, the amount of EVs is an amount collected from about 1.0 x10¹⁰ cells. In some embodiments, the amount of EVs is an amount collected from about 1.0×10¹¹ cells. In some embodiments, the amount of EVs is an amount collected from about 1.0 x10¹² cells. In some embodiments, the amount of EVs is an amount collected from about 1.0×10¹³ cells. In some embodiments, the amount of EVs is an amount collected from about 1.0×10¹⁴ cells. In some embodiments, the amount of EVs is an amount collected from about 1.0×10¹⁵ cells.

### 8.3 Methods of Use of EVs to Cross-Dress Tissues

As used herein, the term “cross-dressing” describes the expression of a transgenic protein (e.g., CD47) in a cell that is induced by, for example, incubating the cell with cells or EVs expressing said protein. For example, porcine cells may be cross-dressed with human CD47 by exposure to cells expressing human CD47, e.g., by co-incubation. In certain embodiments, EVs provided herein are incubated with bone marrow tissue from a donor swine. In some embodiments, EVs provided herein are incubated with a kidney from a donor swine. In some embodiments, EVs provided herein are incubated with both bone marrow tissue and a kidney from the same donor swine. In some embodiments, EVs provided herein are incubated with a kidney from a first donor swine and bone marrow tissue from a second donor swine.

### 8.4 Methods of Use of EVs in Xenotransplantation

Provided herein are methods of xenotransplantation comprising cross-dressing the xenograft with CD47 (e.g., human CD47) prior to implanting the xenograft into the target tissue. In certain embodiments, the target tissue is kidney tissue. In other embodiments, the target tissue is lung tissue. In some embodiments, the target tissue is human. In some embodiments, the target tissue does not express SIRPα (e.g., human SIRPα) as determined by a method known in the art (e.g., Western Blotting, flow cytometry, or quantitative polymerase chain reaction). In certain aspects, the CD47 cross-dressing is independent of Signal Regulatory Protein a (SIRPα) expression on the target tissue.

### 8.4.1. Methods of Exposing Xenografts to EVs

In certain embodiments, the cross-dressing is achieved by exposing the xenograft to EVs comprising CD47. In other embodiments, the cross-dressing is achieved by co-culturing the xenograft with a cell line expressing human CD47 (e.g., a transgenic cell line). In some embodiments, the xenograft is exposed to EVs in vitro. In some embodiments, the xenograft is exposed to EVs in vivo. For example, EVs, such as exosomes, can be injected via the retro-orbital venous sinus, the tail vein or intracardially, or similar approach to deliver the EVs in vivo. In some embodiments, the xenograft is exposed to EVs ex vivo. In specific embodiments, the xenograft vessels are perfused with EVs in vivo. For example, the xenograft can be perfused in vivo in either the donor or the recipient, or both. In some embodiments, the xenograft vessels are perfused with EVs ex vivo.

In some embodiments, the xenograft is exposed to EVs more than once. In some embodiments, the xenograft is exposed to EVs 2, 3, 4, 5, 6, 7, 8, 9 or 10 times over the course of 1, 2, or 3 days. In some embodiments, the xenograft is exposed to the same EV composition repeatedly. In some embodiments, the xenograft is exposed to different EV compositions.

As provided herein, exposure of the xenograft to EVs can occur pre-transplantation, post-transplantation, or both. In some embodiments, the xenograft is exposed to EVs pre-transplantation. In some embodiments, the xenograft is exposed to EVs post-transplantation. In some embodiments, the xenograft is exposed to EVs pre- and post-transplantation.

In some embodiments, the xenograft is exposed post-transplantation more than once. For example, post-transplantation the recipient can be infused (e.g., intravenous infusion) with EVs one or more times. In some embodiments, the recipient is infused daily with EVs. In some embodiments, the recipient is infused more than once daily with EVs. In some embodiments, the recipient is infused.

In specific embodiments, the organ is directly infused in vivo with EVs pre-transplantation, post-transplantation, or a combination thereof. For example, the organ can be perfused directly (e.g., via hepatic portal vein) pre-transplantation. In some embodiments, the organ is directly perfused in vivo pre-transplantation. In some embodiments, the organ is directly perfused in vivo post-transplantation. In some embodiments, the organ is directly perfused in vivo pre-transplantation and post-transplantation. In some embodiments, the organ is directly perfused in vivo more than once pre-transplantation. In some embodiments, the organ is directly perfused in vivo more than once post-transplantation. In some embodiments, the organ is directly perfused in vivo more than once pre-transplantation and post-transplantation.

Exposure of the xenograft to EVs can be for any time sufficient to cross-dress the xenograft. In some embodiments, the xenograft is exposed to EVs for about 1-2 hours, 2-3 hours, 3-4 hours, 4-5 hours, 5-6 hours, 6-7 hours, 7-8 hours, 8-9 hours, 9-10 hours, 10-11 hours or 11-12 hours. In some embodiments, the xenograft is exposed to EVs for about 12-16 hours. In some embodiments, the xenograft is exposed to EVs for about 16-24 hours. In some embodiments, the xenograft is exposed to EVs for more than 24 hours.

In some embodiments, the EVs are engineered to improve their delivery to a specific organ or cell type. Various techniques engineering EVs with targeting properties are known in the art (see, e.g. Murphy, D. E., et al. Exp Mol Med 51, 1-12 (2019)). By way of example, in some embodiments, the EVs express a specific targeting peptide that improves the targeting of EVs to their intended cells of action. Another exemplary targeting technique includes engineering EVs to express a specific integrin combinations that that improves the targeting of EVs to their intended cells of action. In some embodiments, engineering is performed post-EV production.

### 8.4.2. Effects of Exposure to EVs on Xenografts

In some embodiments, cross-dressing of CD47 on cells results in said cell evading phagocytosis. In specific embodiments, cross-dressing of CD47 on cells results in said cells evading phagocytosis without induction of apoptosis.

In certain embodiments, CD47 cross-dressed cells generated according to the present disclosure, such as by the methods described in Section 6.4, express CD47 having decreased ligation with CD47 ligands (e.g., thrombospondin (TSP-1)) relative to a cell expressing CD47 that have not been cross-dressed with CD47 from EVs. In some embodiments, CD47 cross-dressed cells generated according to the present disclosure, such as by the methods described in Section 6.4, express CD47 having no or undetectable levels of ligation with CD47 ligands (e.g., thrombospondin (TSP-1) or SIRPα) relative to a cell expressing CD47 that have not been cross-dressed with CD47 from EVs.

CD47 binding to TSP-1, for example, can cause inflammation and death on a CD47-expressing cell. However, the present disclosure is based in part on findings that CD47 cross-dressed cells do not transmit apoptotic signaling. Conversely, cells that have not been cross-dressed with CD47 and endogenously or exogenously express CD47 do undergo cell death and have increased inflammation. Accordingly, in some embodiments CD47 cross-dressed cells generated according to the present disclosure, exhibit decreased cell death, relative to a cell expressing CD47 that have not been cross-dressed with CD47 from EVs. In certain embodiments, CD47 cross-dressed cells generated according to the present disclosure, exhibit decreased cell death upon exposure to SIRPα or fragments, chimeras, and/or fusion thereof, relative to a cell expressing CD47 that have not been cross-dressed with CD47 from EVs. In specific embodiments, CD47 cross-dressed cells generated according to the present disclosure, exhibit decreased cell death upon exposure to about 50 nM human SIRPα-Fc for 1 hr, relative to a CD47 expressing cell not cross-dressed with CD47 from EVs and exposed to the same amount of SIRPα-Fc.

Phagocytosis can be determined by any method known in the art or described herein, e.g., described in Example 4. For example, cells may be labelled with Celltrace violet and incubated with human macrophages. The level of phagocytosis can then be measured by using flow cytometry to determine the percentage of macrophages (CD14-positive cells) that have engulfed labeled target cells. Provided herein is a method of cross-dressing a xenograft comprising exposing the xenograft to EVs comprising human CD47 prior to transplantation, wherein the method decreases phagocytosis of the xenograft by about 5%, 10%, 15%, 20%, 25%, 30%, 35%, 40%, 45%, 50%, 55%, 60%, 65%, 70%, 75%, 80%, 85%, 90%, or 95% compared to a non-cross-dressed xenograft.

In certain embodiments, the method xenotransplantation provided herein results in decreased phagocytosis without the induction of apoptosis. Apoptosis may be measured using any method known in the art or a method described herein. For example, apoptosis may be measured by staining cell with Propidium iodide (PI) or with PI and Annexin V. Apoptosis may be undetectable or may be decreased by about 5%, 10%, 15%, 20%, 25%, 30%, 35%, 40%, 45%, 50%, 55%, 60%, 65%, 70%, 75%, 80%, 85%, 90%, or 95% compared to a xenograft expressing allogenic CD47.

Evasion of phagocytosis may result in longer survival of a cell and, by extension, prolonged chimerism. For example, cross-dressing of human CD47 in a porcine bone marrow cell may enable the porcine bone marrow cell to evade phagocytosis after transplantation into a human recipient, which results in prolonged survival of the porcine bone marrow cell. Longer survival of the porcine bone marrow cell may then in turn lead to prolonged chimerism, which can be beneficial for avoiding transplant rejection.

As provided herein, cross-dressing a xenograft with EVs comprising human CD47 prior to transplantation reduces inflammation of the xenograft. According, in some embodiments, inflammation of the xenograft may be undetectable or may be decreased by about 5%, 10%, 15%, 20%, 25%, 30%, 35%, 40%, 45%, 50%, 55%, 60%, 65%, 70%, 75%, 80%, 85%, 90%, or 95%, compared to a xenograft expressing allogenic CD47.

Also provided herein, cross-dressing a xenograft with EVs comprising human CD47 prior to transplantation can reduce systemic inflammation in the recipient. According, in some embodiments, systemic inflammation in the recipient post-transplantation of the xenograft may be undetectable or may be decreased by about 5%, 10%, 15%, 20%, 25%, 30%, 35%, 40%, 45%, 50%, 55%, 60%, 65%, 70%, 75%, 80%, 85%, 90%, or 95%, compared to post-transplantation with a xenograft expressing allogenic CD47.

Transplant rejection is a major problem for many recipients of xenografts, which often requires long-term administration of immunosuppressive therapy and evasion of phagocytosis may reduce rejection of xenografts in a recipient. Thus, in one aspect, provided herein are methods for reducing rejection of xenografts in a recipient, the method comprising exposing the xenograft to EVs comprising human CD47 prior to transplantation.

In some embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to a recipient of a xenograft which has not been exposed to EVs comprising human CD47 prior to transplantation. In specific embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the amount of immunosuppressive therapy which is typically administered to a comparable recipient (e.g., a person of the same sex and of comparable age, height, and/or weight who received the same type of tissue or organ as the recipient), wherein the comparable recipient has received a xenograft that has not been exposed to EVs comprising human CD47. In specific embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the standard of care in a comparable clinical setting. In other embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the amount of immunosuppressive therapy which said recipient required after receipt of a prior xenograft, wherein the prior xenograft was not exposed to EVs comprising human CD47 prior to transplantation. In other embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the amount of immunosuppressive therapy required in a comparable clinical setting, wherein the xenograft in the comparable clinical setting was not exposed to EVs comprising human CD47 prior to transplantation. In some embodiments, method results in the recipient requiring no further administration of immunosuppressive therapy, e.g., an immunosuppressive therapy described in section 6.4.3 below.

In some embodiments, the method results in prolonged viability of the xenograft compared to a xenograft that has not been exposed to EVs comprising human CD47 prior to transplantation. In some embodiments, the method results in prolonged viability (e.g., viability prolonged about 10%, 10-20%, 20-30%, 30-40%, 40-50%, 50-75%, 75-100%, 100-200%, 200-300% or by over 300%; or prolonged by 1-2 years, 2-3 years, 3-4 years, 4-5 years, 5-6 years, 6-8 years, 8-10 years, 10-15 years or 15-20 years) of the xenograft compared to a comparable xenograft (e.g., the same type of tissue or organ as the recipient) transplanted into a comparable recipient (e.g., a patient of the same sex and of comparable age, height, and/or weight), wherein the comparable xenograft has not been exposed to EVs comprising human CD47 prior to transplantation. In some embodiments, the method results in prolonged viability (e.g., viability prolonged about 10%, 10-20%, 20-30%, 30-40%, 40-50%, 50-75%, 75-100%, 100-200%, 200-300% or by over 300%; or prolonged by 1-2 years, 2-3 years, 3-4 years, 4-5 years, 5-6 years, 6-8 years, 8-10 years, 10-15 years or 15-20 years) of the xenograft compared to the viability of a xenograft which said recipient has previously received, wherein the xenograft previously received was not exposed to EVs comprising human CD47 prior to transplantation.

In some embodiments, the method results in prolonged viability (e.g., viability prolonged about 10%, 10-20%, 20-30%, 30-40%, 40-50%, 50-75%, 75-100%, 100-200%, 200-300% or by over 300%; or prolonged by 1-2 years, 2-3 years, 3-4 years, 4-5 years, 5-6 years, 6-8 years, 8-10 years, 10-15 years or 15-20 years) compared to the viability of a xenograft in a comparable clinical setting, wherein the xenograft in the comparable clinical setting was not exposed to EVs comprising human CD47 prior to transplantation.

In some embodiments, the method results in better health-related quality of life for the recipient compared to a recipient of a xenograft that has not been exposed to EVs comprising human CD47 prior to transplantation. In other embodiments, the method results in better health-related quality of life for the recipient compared to a comparable recipient (e.g., a person of the same sex and of comparable age, height, and/or weight who received the same type of tissue or organ as the recipient), wherein the comparable recipient has received a xenograft that has not been exposed to EVs comprising human CD47 prior to transplantation. In other embodiments, the method results in better health-related quality of life for the recipient compared to the health-related quality of life said recipient experienced after a prior xenotransplantation. In other embodiments, the method results in better health-related quality of life for the recipient compared to a comparable clinical setting, wherein the xenograft in the comparable clinical setting has not been exposed to EVs comprising human CD47 prior to transplantation. Health-related quality of life refers to the overall impact of health aspects on an individual's quality of life and includes physical symptoms, functional status, psychological states, and social relationships. Health-related quality of life may be assessed by any instrument known in the art, including, for example, the 36-Item Short Form Survey (SF-36), the EuroQol—5 Dimensions (EQ-5D) and the Kidney Disease Quality of Life Instrument (KDQOL). See, e.g., Parizi et al. The Patient—Patient-Centered Outcomes Research (2019) 12:171-181.

In some embodiments, the method results in longer survival (e.g., 10-20%, 20-30%, 40-50%, 50-60%, 60-70%, 70-80%, 80-90%, or 90-100% longer; or 2 to 3-fold, 3 to 5-fold, 5 to 7-fold, 7 to 10-fold or 10 to 15-fold longer) of the transplant recipient compared to a recipient of a xenograft which has not been exposed to EVs comprising human CD47 prior to transplantation. In other embodiments, the method results in longer survival (e.g., 10-20%, 20-30%, 40-50%, 50-60%, 60-70%, 70-80%, 80-90%, or 90-100% longer; or 2 to 3-fold, 3 to 5-fold, 5 to 7-fold, 7 to 10-fold or 10 to 15-fold longer) of the transplant recipient compared to the survival of a comparable recipient (e.g., a person of the same sex and of comparable age, height, and/or weight who received the same type of tissue or organ as the recipient), wherein the comparable recipient has received a xenograft that has not been exposed to EVs comprising human CD47. In other embodiments, the method results in longer survival (e.g., 10-20%, 20-30%, 40-50%, 50-60%, 60-70%, 70-80%, 80-90%, or 90-100% longer; or 2 to 3-fold, 3 to 5-fold, 5 to 7-fold, 7 to 10-fold or 10 to 15-fold longer) of the transplant recipient compared to the survival of a transplant recipient in a comparable clinical setting, wherein the xenograft in the comparable clinical setting has not been exposed to EVs comprising human CD47 prior to transplantation.

In one aspect, the methods of transplantation described herein result in reduced risk, severity or duration of proteinuria. Protein excretion of more than 150 mg per day is a commonly a used as a diagnosis for proteinuria. Dipstick analysis is often used to measure protein concentrations in the urine. This is a semi-quantitative method, the results of which are expressed as negative, trace, 1+, 2+, 3+ or 4+. See e.g., Carroll and Temte, Am Fam Physician 62 (6): 1333-1340 (2000). Total protein levels or only albumin levels may be measured to provide a quantitative test. Results may be expressed in total protein or albumin levels, or in alumni to creatine ration or protein to creatine ratio.

In particular embodiments, the methods of transplantation described herein result in a reduced severity of proteinuria. In particular embodiments, the methods of transplantation described herein result in a reduced duration of proteinuria. For example, the severity of proteinuria in a patient treated in accordance with the methods herein may be decreased compared to the severity of proteinuria observed in a patient receiving a donor kidney wherein the donor kidney has not been cross-dressed with human CD47.

In some embodiments, the severity of proteinuria, as measured by protein levels in the urine, is reduced by 10%, 20%, 30%, 40%, 50%, 60%, 70%, 80%, 90%, 95% or over 95%. In some embodiments, a patient treated in accordance with a method provided herein will not experience proteinuria, defined as the excretion or over 150 mg protein per day in the urine. In some embodiments, a patient treated in accordance with a method provided herein may experience transient proteinuria that resolves after 1, 2, 3, 3-7, 7-10, 10-14 days, or 1-2, 2-3, 3-4, 4-5, 5-6, 6-7, 7-8 weeks, or 1, 2, 3, 4, 5, 6 months after the transplantation.

In some embodiments, the concentration of total protein in the urine of a recipient treated with a method described herein developing proteinuria is less than about 60 mg per day, less than about 80 mg per day, less than about 100 mg per day, less than about 120 mg per day, less than about 140 mg per day, less than about 160 mg per day, less than about 200 mg per day, less than about 220 mg per day, less than about 240 mg, per day, less than about 260 mg per day, less than about 280 mg per day, less than about 300 mg per day, less than about 320 mg per day, less than about 340 mg per day, less than about 360 mg per day, less than about 380 mg per day or less than about 400 mg per day.

In some embodiments, the concentration of albumin in the urine of a recipient treated with a method described herein developing proteinuria is less than about 5 mg per day, less than about 10 mg per day, less than about 20 mg per day, less than about 30 mg per day, less than about 40 mg per day, less than about 50 mg per day, less than about 60 mg per day, less than about 70 mg per day, less than about 80 mg per day, less than about 90 mg per day or less than about 100 mg per day.

In some embodiments, the ratio of protein to creatinine in a 24 hour urine sample of a patient treated in accordance with the methods described herein is less than about 0.2, less than about 0.4, less than about 0.6, less than about 0.8 or less than about 1. In some embodiments, the ratio of albumin to creatinine in a 24 hour urine sample of a patient treated in accordance with the methods described herein is less than about 0.02, less than about 0.04, less than about 0.06, less than about 0.08 or less than about 0.1.

In some embodiments, the risk of a recipient treated with a method described herein developing proteinuria is decreased by about 10%, 20%, 30%, 40%, 50%, 60%, 70%, 80%, 90% or 95% compared to the risk of a recipient of a donor kidney wherein the donor kidney has not been cross-dressed with human CD47.

### 8.4.3. Additional Treatments

In certain aspect, a patient treated in accordance with the methods described herein undergoes additional treatment. A patient may undergo additional treatment by one or more difference methods. Additional treatment may occur prior to, concurrently with, or subsequent to the method of treatment provided herein.

In certain embodiments, a patient receiving a xenograft in accordance with the methods described herein receives an intra-bone bone marrow transplantation (IBBM). In specific embodiments, the bone marrow is from the same source as the xenograft. In specific embodiments, the bone marrow expresses human CD47.

In some embodiments, the patient receiving a xenograft in accordance with the methods described herein receives immunosuppressive therapy. The immunosuppressive therapy may be any FDA-approved treatment indicated to reduce transplant rejection and/or ameliorate the outcome of xenotransplantation. Non-limiting examples of immunosuppressive therapy include calcineurin inhibitors (e.g., tacrolimus or cyclosporine), antiproliferative agents (e.g., anti-metabolites such a mycophenolate, 6-mercaptopurine or its prodrug azathioprine), inhibitors of mammalian target of rapamycin (mTOR) (e.g., sirolimus, rapamycin), steroids (e.g., prednisone), cell cycle inhibitors (azathioprine or mycophenolate mofetil), lymphocyte-depleting agents (e.g., anti-thymocyte globulin or antibodies such as alemtuzumab, siplizumab or basiliximab) and co-stimulation blockers (e.g., belatacept). See, e.g., Chung et al (2020)., Ann Transl Med. March; 8 (6): 409; van der Mark et al. (2020), Eur Respir Rev; 29:190132 and Benvenuto et al. (2018), J Thorac Dis 10:3141-3155.

Immunosuppressive therapy may be administered as induction therapy (perioperative, or immediately after surgery) a maintenance dose or for an acute rejection. Induction therapy commonly includes basiliximab, anti-thymocyte globulin or alemtuzumab. Immunosuppressive therapy may also be administered as maintenance therapy which is often required to continue for the life of the recipient. Maintenance immunosuppressive therapy commonly includes a calcineurin inhibitor (tacrolimus or cyclosporine), an antiproliferative agent (mycophenolate or azathioprine), and corticosteroids. Immunosuppressive therapy for acute rejections commonly includes thymoglobulin or mycophenolate. See, e.g., Chung et al. (2020), Ann Transl Med. March; 8:409 and Benvenuto et al., (2018) J Thorac Dis 10:3141-3155.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CDI la (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizumab, inolimomab).

### 8.4.4. Patient Population

In a preferred embodiment, a patient treated in accordance with the methods described herein (e.g., the recipient of a xenograft that has been cross-dressed with human CD47) is a human patient. As used herein, the terms “subject” and “patient” are used interchangeably and include any human or non-human mammal. Non-limiting examples include members of the human, equine, porcine, bovine, rattus, murine, canine and feline species. In some embodiments, the subject is a non-human primate. In some embodiments, the subject is human. In specific embodiments, the subject is a human adult. In some embodiments, the subject is a human child. In specific embodiments, the subject is human and receives one or more donor grafts from a porcine donor. In other specific embodiments, the subject is a non-human primate (e.g., a baboon, a cynomolgus monkey or a rhesus macaque) and receives one or more grafts from a porcine donor.

In one aspect, a patient treated in accordance with the methods described herein is in need of a kidney transplant. A patient may be in need of a kidney transplant due to renal failure or the rejection of a donor kidney. Renal failure can have a number of causes, including but not limited to high blood pressure (hypertension), physical injury, diabetes, kidney disease (polycystic kidney disease, glomerular disease) and autoimmune disorders such as lupus. Renal failure may be acute or chronic. Kidney failure can also be diagnosed by laboratory tests such as glomerular filtration rate, blood urea nitrogen, and serum creatinine, by imaging test (ultrasound, computer tomography) or a kidney biopsy.

In some embodiments, a patient treated in accordance with a method described herein has Stage 1 kidney disease. In some embodiments, a patient treated in accordance with a method described herein has Stage 2 kidney disease. In some embodiments, a patient treated in accordance with a method described herein has Stage 3 kidney disease. In some embodiments, a patient treated in accordance with a method described herein has Stage 4 kidney disease. In some embodiments, a patient treated in accordance with a method described herein has Stage 5 kidney disease.

In some embodiments, a patient treated in accordance with a method described herein has a glomerular filtration rate (GFR) of about 90 or higher. In some embodiments, a patient treated in accordance with a method described herein has a GFR of about 60-90. In some embodiments, a patient treated in accordance with a method described herein has a GFR of about 30-60. In some embodiments, a patient treated in accordance with a method described herein has a GFR of about 15-30. In some embodiments, a patient treated in accordance with a method described herein has a GFR of about 15 or less.

TABLE 1
Table of Sequences

| Name | SEQ ID NO: | Sequence |
|---|---|---|
| leukocyte surface | 1 | MWPLVAALLLGSACCGSAQLLFNKTKSVEFTFCNDT |
| antigen CD47 isoform | | VVIPCFVTNMEAQNTTEVYVKWKFKGRDIYTFDGAL |
| 1 precursor [Homo | | NKSTVPTDFSSAKIEVSQLLKGDASLKMDKSDAVSHT |
| sapiens] | | GNYTCEVTELTREGETIIELKYRVVSWFSPNENILIVIF |
| (NCBI Reference | | PIFAILLFWGQFGIKTLKYRSGGMDEKTIALLVAGLVI |
| Sequence: | | TVIVIVGAILFVPGEYSLKNATGLGLIVTSTGILILLHY |
| NP_001768.1 | | YVFSTAIGLTSFVIAILVIQVIAYILAVVGLSLCIAACIP |
| | | MHGPLLISGLSILALAQLLGLVYMKFVASNQKTIQPP |
| | | RKAVEEPLNAFKESKGMMNDE |
| CD47 molecule [Homo | 2 | MWPLVAALLLGSACCGSAQLLFNKTKSVEFTFCNDT |
| sapiens (human)] | | VVIPCFVTNMEAQNTTEVYVKWKFKGRDIYTFDGAL |
| NCBI Reference | | NKSTVPTDFSSAKIEVSQLLKGDASLKMDKSDAVSHT |
| Sequence: NP_942088 | | GNYTCEVTELTREGETIIELKYRVVSWFSPNENILIVIF |
| | | PIFAILLFWGQFGIKTLKYRSGGMDEKTIALLVAGLVI |
| | | TVIVIVGAILFVPGEYSLKNATGLGLIVTSTGILILLHY |
| | | YVFSTAIGLTSFVIAILVIQVIAYILAVVGLSLCIAACIP |
| | | MHGPLLISGLSILALAQLLGLVYMKFVASNQKTIQPP |
| | | RNN |
| leukocyte surface | 3 | MWPLVAALLLGSACCGSAQLLFNKTKSVEFTFCNDT |
| antigen CD47 isoform | | VVIPCFVTNMEAQNTTEVYVKWKFKGRDIYTFDGAL |
| 3 precursor [Homo | | NKSTVPTDFSSAKIEVSQLLKGDASLKMDKSDAVSHT |
| sapiens] | | GNYTCEVTELTREGETIIELKYRVVSWFSPNENILIVIF |
| NCBI Reference | | PIFAILLFWGQFGIKTLKYRSGGMDEKTIALLVAGLVI |
| Sequence: | | TVIVIVGAILFVPGEYSLKNATGLGLIVTSTGILILLHY |
| NP_001369235.1 | | YVFSTAIGLTSFVIAILVIQVIAYILAVVGLSLCIAACIP |
| | | MHGPLLISGLSILALAQLLGLVYMKFVASNQKTIQPP |
| | | RKAVEEPLNE |
| Homo sapiens | 4 | MWPLVAALLLGSACCGSAQLLFNKTKSVEFTFCNDT |
| leukocyte surface | | VVIPCFVTNMEAQNTTEVYVKWKFKGRDIYTFDGAL |
| antigen CD47 isoform | | NKSTVPTDFSSAKIEVSQLLKGDASLKMDKSDAVSHT |
| X2 | | GNYTCEVTELTREGETIIELKYRVVSWFSPNENILIVIF |
| NCBI Reference | | PIFAILLFWGQFGIKTLKYRSGGMDEKTIALLVAGLVI |
| Sequence: | | TVIVIVGAILFVPGEYSLKNATGLGLIVTSTGILILLHY |
| XP_005247966.1 | | YVFSTAIGLTSFVIAILVIQVIAYILAVVGLSLCIAACIP |
| | | MHGPLLISGLSILALAQLLGLVYMKFVE |
| Homo sapiens CD47 | 5 | GCAGCCTGGGCAGTGGGTCCTGCCTGTGACGCGCG |
| molecule (CD47), | | GCGGCGGTCGGTCCTGCCTGTAACGGCGGCGGCGG |
| transcript variant 1, | | CTGCTGCTCCGGACACCTGCGGCGGCGGCGGCGAC |
| mRNA | | CCCGCGGCGGGCGCGGAGATGTGGCCCCTGGTAGC |
| NCBI Reference | | GGCGCTGTTGCTGGGCTCGGCGTGCTGCGGATCAG |
| Sequence: NM_001777 | | CTCAGCTACTATTTAATAAAACAAAATCTGTAGAA |
| | | TTCACGTTTTGTAATGACACTGTCGTCATTCCATGC |
| | | TTTGTTACTAATATGGAGGCACAAAACACTACTGA |
| | | AGTATACGTAAAGTGGAAATTTAAAGGAAGAGATA |
| | | TTTACACCTTTGATGGAGCTCTAAACAAGTCCACTG |
| | | TCCCCACTGACTTTAGTAGTGCAAAAATTGAAGTCT |
| | | CACAATTACTAAAAGGAGATGCCTCTTTGAAGATG |
| | | GATAAGAGTGATGCTGTCTCACACACAGGAAACTA |
| | | CACTTGTGAAGTAACAGAATTAACCAGAGAAGGTG |
| | | AAACGATCATCGAGCTAAAATATCGTGTTGTTTCAT |
| | | GGTTTTCTCCAAATGAAAATATTCTTATTGTTATTT |
| | | TCCCAATTTTTGCTATACTCCTGTTCTGGGGACAGT |
| | | TTGGTATTAAAACACTTAAATATAGATCCGGTGGT |
| | | ATGGATGAGAAAACAATTGCTTTACTTGTTGCTGG |
| | | ACTAGTGATCACTGTCATTGTCATTGTTGGAGCCAT |
| | | TCTTTTCGTCCCAGGTGAATATTCATTAAAGAATGC |
| | | TACTGGCCTTGGTTTAATTGTGACTTCTACAGGGAT |
| | | ATTAATATTACTTCACTACTATGTGTTTAGTACAGC |
| | | GATTGGATTAACCTCCTTCGTCATTGCCATATTGGT |
| | | TATTCAGGTGATAGCCTATATCCTCGCTGTGGTTGG |
| | | ACTGAGTCTCTGTATTGCGGCGTGTATACCAATGCA |
| | | TGGCCCTCTTCTGATTTCAGGTTTGAGTATCTTAGC |
| | | TCTAGCACAATTACTTGGACTAGTTTATATGAAATT |
| | | TGTGGCTTCCAATCAGAAGACTATACAACCTCCTA |
| | | GGAAAGCTGTAGAGGAACCCCTTAATGCATTCAAA |
| | | GAATCAAAAGGAATGATGAATGATGAATAACTGA |
| | | AGTGAAGTGATGGACTCCGATTTGGAGAGTAGTAA |
| | | GACGTGAAAGGAATACACTTGTGTTTAAGCACCAT |
| | | GGCCTTGATGATTCACTGTTGGGGAGAAGAAACAA |
| | | GAAAAGTAACTGGTTGTCACCTATGAGACCCTTAC |
| | | GTGATTGTTAGTTAAGTTTTTATTCAAAGCAGCTGT |
| | | AATTTAGTTAATAAAATAATTATGATCTATGTTGTT |
| | | TGCCCAATTGAGATCCAGTTTTTTGTTGTTATTTTTA |
| | | ATCAATTAGGGGCAATAGTAGAATGGACAATTTCC |
| | | AAGAATGATGCCTTTCAGGTCCTAGGGCCTCTGGC |
| | | CTCTAGGTAACCAGTTTAAATTGGTTCAGGGTGAT |
| | | AACTACTTAGCACTGCCCTGGTGATTACCCAGAGA |
| | | TATCTATGAAAACCAGTGGCTTCCATCAAACCTTTG |
| | | CCAACTCAGGTTCACAGCAGCTTTGGGCAGTTATG |
| | | GCAGTATGGCATTAGCTGAGAGGTGTCTGCCACTT |
| | | CTGGGTCAATGGAATAATAAATTAAGTACAGGCAG |
| | | GAATTTGGTTGGGAGCATCTTGTATGATCTCCGTAT |
| | | GATGTGATATTGATGGAGATAGTGGTCCTCATTCTT |
| | | GGGGGTTGCCATTCCCACATTCCCCCTTCAACAAAC |
| | | AGTGTAACAGGTCCTTCCCAGATTTAGGGTACTTTT |
| | | ATTGATGGATATGTTTTCCTTTTATTCACATAACCC |
| | | CTTGAAACCCTGTCTTGTCCTCCTGTTACTTGCTTCT |
| | | GCTGTACAAGATGTAGCACCTTTTCTCCTCTTTGAA |
| | | CATGGTCTAGTGACACGGTAGCACCAGTTGCAGGA |
| | | AGGAGCCAGACTTGTTCTCAGAGCACTGTGTTCAC |
| | | ACTTTTCAGCAAAAATAGCTATGGTTGTAACATAT |
| | | GTATTCCCTTCCTCTGATTTGAAGGCAAAAATCTAC |
| | | AGTGTTTCTTCACTTCTTTTCTGATCTGGGGCATGA |
| | | AAAAAGCAAGATTGAAATTTGAACTATGAGTCTCC |
| | | TGCATGGCAACAAAATGTGTGTCACCATCAGGCCA |
| | | ACAGGCCAGCCCTTGAATGGGGATTTATTACTGTT |
| | | GTATCTATGTTGCATGATAAACATTCATCACCTTCC |
| | | TCCTGTAGTCCTGCCTCGTACTCCCCTTCCCCTATG |
| | | ATTGAAAAGTAAACAAAACCCACATTTCCTATCCT |
| | | GGTTAGAAGAAAATTAATGTTCTGACAGTIGTGAT |
| | | CGCCTGGAGTACTTTTAGACTTTTAGCATTCGTTTT |
| | | TTACCTGTTTGTGGATGTGTGTTTGTATGTGCATAC |
| | | GTATGAGATAGGCACATGCATCTTCTGTATGGACA |
| | | AAGGTGGGGTACCTACAGGAGAGCAAAGGTTAATT |
| | | TTGTGCTTTTAGTAAAAACATTTAAATACAAAGTTC |
| | | TTTATTGGGTGGAATTATATTTGATGCAAATATTTG |
| | | ATCACTTAAAACTTTTAAAACTTCTAGGTAATTTGC |
| | | CACGCTTTTTGACTGCTCACCAATACCCTGTAAAAA |
| | | TACGTAATTCTTCCTGTTTGTGTAATAAGATATTCA |
| | | TATTTGTAGTTGCATTAATAATAGTTATTTCTTAGT |
| | | CCATCAGATGTTCCCGTGTGCCTCTTTTATGCCAAA |
| | | TTGATTGTCATATTTCATGTTGGGACCAAGTAGTTT |
| | | GCCCATGGCAAACCTAAATTTATGACCTGCTGAGG |
| | | CCTCTCAGAAAACTGAGCATACTAGCAAGACAGCT |
| | | CTTCTTGAAAAAAAAAATATGTATACACAAATATA |
| | | TACGTATATCTATATATACGTATGTATATACACACA |
| | | TGTATATTCTTCCTTGATTGTGTAGCTGTCCAAAAT |
| | | AATAACATATATAGAGGGAGCTGTATTCCTTTATA |
| | | CAAATCTGATGGCTCCTGCAGCACTTTTTCCTTCTG |
| | | AAAATATTTACATTTTGCTAACCTAGTTTGTTACTT |
| | | TAAAAATCAGTTTTGATGAAAGGAGGGAAAAGCA |
| | | GATGGACTTGAAAAAGATCCAAGCTCCTATTAGAA |
| | | AAGGTATGAAAATCTTTATAGTAAAATTTTTTATAA |
| | | ACTAAAGTTGTACCTTTTAATATGTAGTAAACTCTC |
| | | ATTTATTTGGGGTTCGCTCTTGGATCTCATCCATCC |
| | | ATTGTGTTCTCTTTAATGCTGCCTGCCTTTTGAGGC |
| | | ATTCACTGCCCTAGACAATGCCACCAGAGATAGTG |
| | | GGGGAAATGCCAGATGAAACCAACTCTTGCTCTCA |
| | | CTAGTTGTCAGCTTCTCTGGATAAGTGACCACAGA |
| | | AGCAGGAGTCCTCCTGCTTGGGCATCATTGGGCCA |
| | | GTTCCTTCTCTTTAAATCAGATTTGTAATGGCTCCC |
| | | AAATTCCATCACATCACATTTAAATTGCAGACAGT |
| | | GTTTTGCACATCATGTATCTGTTTTGTCCCATAATA |
| | | TGCTTTTTACTCCCTGATCCCAGTTTCTGCTGTTGAC |
| | | TCTTCCATTCAGTTTTATTTATTGTGTGTTCTCACAG |
| | | TGACACCATTTGTCCTTTTCTGCAACAACCTTTCCA |
| | | GCTACTTTTGCCAAATTCTATTTGTCTTCTCCTTCAA |
| | | AACATTCTCCTTTGCAGTTCCTCTTCATCTGTGTAG |
| | | CTGCTCTTTTGTCTCTTAACTTACCATTCCTATAGTA |
| | | CTTTATGCATCTCTGCTTAGTTCTATTAGTTTTTTGG |
| | | CCTTGCTCTTCTCCTTGATTTTAAAATTCCTTCTATA |
| | | GCTAGAGCTTTTCTTTCTTTCATTCTCTCTTCCTGCA |
| | | GTGTTTTGCATACATCAGAAGCTAGGTACATAAGT |
| | | TAAATGATTGAGAGTTGGCTGTATTTAGATTTATCA |
| | | CTTTTTAATAGGGTGAGCTTGAGAGTTTTCTTTCTT |
| | | TCTGTTTTTTTTTTTTGTTTTTTTTTTTTTTTTTTTTTT |
| | | TTTTTTTTTTGACTAATTTCACATGCTCTAAAAACCT |
| | | TCAAAGGTGATTATTTTTCTCCTGGAAACTCCAGGT |
| | | CCATTCTGTTTAAATCCCTAAGAATGTCAGAATTAA |
| | | AATAACAGGGCTATCCCGTAATTGGAAATATTTCTT |
| | | TTTTCAGGATGCTATAGTCAATTTAGTAAGTGACCA |
| | | CCAAATTGTTATTTGCACTAACAAAGCTCAAAACA |
| | | CGATAAGTTTACTCCTCCATCTCAGTAATAAAAATT |
| | | AAGCTGTAATCAACCTTCTAGGTTTCTCTTGTCTTA |
| | | AAATGGGTATTCAAAAATGGGGATCTGTGGTGTAT |
| | | GTATGGAAACACATACTCCTTAATTTACCTGTTGTT |
| | | GGAAACTGGAGAAATGATTGTCGGGCAACCGTTTA |
| | | TTTTTTATTGTATTTTATTTGGTTGAGGGATTTTTTT |
| | | ATAAACAGTTTTACTTGTGTCATATTTTAAAATTAC |
| | | TAACTGCCATCACCTGCTGGGGTCCTTTGTTAGGTC |
| | | ATTTTCAGTGACTAATAGGGATAATCCAGGTAACT |
| | | TTGAAGAGATGAGCAGTGAGTGACCAGGCAGTTTT |
| | | TCTGCCTTTAGCTTTGACAGTTCTTAATTAAGATCA |
| | | TTGAAGACCAGCTTTCTCATAAATTTCTCTTTTTGA |
| | | AAAAAAGAAAGCATTTGTACTAAGCTCCTCTGTAA |
| | | GACAACATCTTAAATCTTAAAAGTGTTGTTATCATG |
| | | ACTGGTGAGAGAAGAAAACATTTTGTTTTTATTAA |
| | | ATGGAGCATTATTTACAAAAAGCCATTGTTGAGAA |
| | | TTAGATCCCACATCGTATAAATATCTATTAACCATT |
| | | CTAAATAAAGAGAACTCCAGTGTTGCTATGTGCAA |
| | | GATCCTCTCTTGGAGCTTTTTTGCATAGCAATTAAA |
| | | GGTGTGCTATTTGTCAGTAGCCATTTTTTTGCAGTG |
| | | ATTTGAAGACCAAAGTTGTTTTACAGCTGTGTTACC |
| | | GTTAAAGGTTTTTTTTTTTATATGTATTAAATCAATT |
| | | TATCACTGTTTAAAGCTTTGAATATCTGCAATCTTT |
| | | GCCAAGGTACTTTTTTATTTAAAAAAAAACATAAC |
| | | TTTGTAAATATTACCCTGTAATATTATATATACTTA |
| | | ATAAAACATTTTAAGCTATTTTGTTGGGCTATTTCT |
| | | ATTGCTGCTACAGCAGACCACAAGCACATTTCTGA |
| | | AAAATTTAATTTATTAATGTATTTTTAAGTTGCTTA |
| | | TATTCTAGGTAACAATGTAAAGAATGATTTAAAAT |
| | | ATTAATTATGAATTTTTTGAGTATAATACCCAATAA |
| | | GCTTTTAATTAGAGCAGAGTTTTAATTAAAAGTTTT |
| | | AAATCAGTCCAA |
| Homo sapiens CD47 | 6 | GGGGAGCAGGCGGGGGAGCGGGCGGGAAGCAGTG |
| molecule (CD47), | | GGAGCGCGCGTGCGCGCGGCCGTGCAGCCTGGGCA |
| transcript variant 2, | | GTGGGTCCTGCCTGTGACGCGCGGCGGCGGTCGGT |
| mRNA | | CCTGCCTGTAACGGCGGCGGCGGCTGCTGCTCCAG |
| NCBI Reference | | ACACCTGCGGCGGCGGCGGCGACCCCGCGGCGGGC |
| Sequence: | | GCGGAGATGTGGCCCCTGGTAGCGGCGCTGTTGCT |
| NM_198793.3 | | GGGCTCGGCGTGCTGCGGATCAGCTCAGCTACTAT |
| | | TTAATAAAACAAAATCTGTAGAATTCACGTTTTGTA |
| | | ATGACACTGTCGTCATTCCATGCTTTGTTACTAATA |
| | | TGGAGGCACAAAACACTACTGAAGTATACGTAAAG |
| | | TGGAAATTTAAAGGAAGAGATATTTACACCTTTGA |
| | | TGGAGCTCTAAACAAGTCCACTGTCCCCACTGACTT |
| | | TAGTAGTGCAAAAATTGAAGTCTCACAATTACTAA |
| | | AAGGAGATGCCTCTTTGAAGATGGATAAGAGTGAT |
| | | GCTGTCTCACACACAGGAAACTACACTTGTGAAGT |
| | | AACAGAATTAACCAGAGAAGGTGAAACGATCATC |
| | | GAGCTAAAATATCGTGTTGTTTCATGGTTTTCTCCA |
| | | AATGAAAATATTCTTATTGTTATTTTCCCAATTTTT |
| | | GCTATACTCCTGTTCTGGGGACAGTTTGGTATTAAA |
| | | ACACTTAAATATAGATCCGGTGGTATGGATGAGAA |
| | | AACAATTGCTTTACTTGTTGCTGGACTAGTGATCAC |
| | | TGTCATTGTCATTGTTGGAGCCATTCTTTTCGTCCC |
| | | AGGTGAATATTCATTAAAGAATGCTACTGGCCTTG |
| | | GTTTAATTGTGACTTCTACAGGGATATTAATATTAC |
| | | TTCACTACTATGTGTTTAGTACAGCGATTGGATTAA |
| | | CCTCCTTCGTCATTGCCATATTGGTTATTCAGGTGA |
| | | TAGCCTATATCCTCGCTGTGGTTGGACTGAGTCTCT |
| | | GTATTGCGGCGTGTATACCAATGCATGGCCCTCTTC |
| | | TGATTTCAGGTTTGAGTATCTTAGCTCTAGCACAAT |
| | | TACTTGGACTAGTTTATATGAAATTTGTGGCTTCCA |
| | | ATCAGAAGACTATACAACCTCCTAGGAATAACTGA |
| | | AGTGAAGTGATGGACTCCGATTTGGAGAGTAGTAA |
| | | GACGTGAAAGGAATACACTTGTGTTTAAGCACCAT |
| | | GGCCTTGATGATTCACTGTTGGGGAGAAGAAACAA |
| | | GAAAAGTAACTGGTTGTCACCTATGAGACCCTTAC |
| | | GTGATTGTTAGTTAAGTTTTTATTCAAAGCAGCTGT |
| | | AATTTAGTTAATAAAATAATTATGATCTATGTTGTT |
| | | TGCCCAATTGAGATCCAGTTTTTTGTTGTTATTTTTA |
| | | ATCAATTAGGGGCAATAGTAGAATGGACAATTTCC |
| | | AAGAATGATGCCTTTCAGGTCCTAGGGCCTCTGGC |
| | | CTCTAGGTAACCAGTTTAAATTGGTTCAGGGTGAT |
| | | AACTACTTAGCACTGCCCTGGTGATTACCCAGAGA |
| | | TATCTATGAAAACCAGTGGCTTCCATCAAACCTTTG |
| | | CCAACTCAGGTTCACAGCAGCTTTGGGCAGTTATG |
| | | GCAGTATGGCATTAGCTGAGAGGTGTCTGCCACTT |
| | | CTGGGTCAATGGAATAATAAATTAAGTACAGGCAG |
| | | GAATTTGGTTGGGAGCATCTTGTATGATCTCCGTAT |
| | | GATGTGATATTGATGGAGATAGTGGTCCTCATTCTT |
| | | GGGGGTTGCCATTCCCACATTCCCCCTTCAACAAAC |
| | | AGTGTAACAGGTCCTTCCCAGATTTAGGGTACTTTT |
| | | ATTGATGGATATGTTTTCCTTTTATTCACATAACCC |
| | | CTTGAAACCCTGTCTTGTCCTCCTGTTACTTGCTTCT |
| | | GCTGTACAAGATGTAGCACCTTTTCTCCTCTTTGAA |
| | | CATGGTCTAGTGACACGGTAGCACCAGTTGCAGGA |
| | | AGGAGCCAGACTTGTTCTCAGAGCACTGTGTTCAC |
| | | ACTTTTCAGCAAAAATAGCTATGGTTGTAACATAT |
| | | GTATTCCCTTCCTCTGATTTGAAGGCAAAAATCTAC |
| | | AGTGTTTCTTCACTTCTTTTCTGATCTGGGGCATGA |
| | | AAAAAGCAAGATTGAAATTTGAACTATGAGTCTCC |
| | | TGCATGGCAACAAAATGTGTGTCACCATCAGGCCA |
| | | ACAGGCCAGCCCTTGAATGGGGATTTATTACTGTT |
| | | GTATCTATGTTGCATGATAAACATTCATCACCTTCC |
| | | TCCTGTAGTCCTGCCTCGTACTCCCCTTCCCCTATG |
| | | ATTGAAAAGTAAACAAAACCCACATTTCCTATCCT |
| | | GGTTAGAAGAAAATTAATGTTCTGACAGTTGTGAT |
| | | CGCCTGGAGTACTTTTAGACTTTTAGCATTCGTTTT |
| | | TTACCTGTTTGTGGATGTGTGTTTGTATGTGCATAC |
| | | GTATGAGATAGGCACATGCATCTTCTGTATGGACA |
| | | AAGGTGGGGTACCTACAGGAGAGCAAAGGTTAATT |
| | | TTGTGCTTTTAGTAAAAACATTTAAATACAAAGTTC |
| | | TTTATTGGGTGGAATTATATTTGATGCAAATATTTG |
| | | ATCACTTAAAACTTTTAAAACTTCTAGGTAATTTGC |
| | | CACGCTTTTTGACTGCTCACCAATACCCTGTAAAAA |
| | | TACGTAATTCTTCCTGTTTGTGTAATAAGATATTCA |
| | | TATTTGTAGTTGCATTAATAATAGTTATTTCTTAGT |
| | | CCATCAGATGTTCCCGTGTGCCTCTTTTATGCCAAA |
| | | TTGATTGTCATATTTCATGTTGGGACCAAGTAGTTT |
| | | GCCCATGGCAAACCTAAATTTATGACCTGCTGAGG |
| | | CCTCTCAGAAAACTGAGCATACTAGCAAGACAGCT |
| | | CTTCTTGAAAAAAAAAATATGTATACACAAATATA |
| | | TACGTATATCTATATATACGTATGTATATACACACA |
| | | TGTATATTCTTCCTTGATTGTGTAGCTGTCCAAAAT |
| | | AATAACATATATAGAGGGAGCTGTATTCCTTTATA |
| | | CAAATCTGATGGCTCCTGCAGCACTTTTTCCTTCTG |
| | | AAAATATTTACATTTTGCTAACCTAGTTTGTTACTT |
| | | TAAAAATCAGTTTTGATGAAAGGAGGGAAAAGCA |
| | | GATGGACTTGAAAAAGATCCAAGCTCCTATTAGAA |
| | | AAGGTATGAAAATCTTTATAGTAAAATTTTTTATAA |
| | | ACTAAAGTTGTACCTTTTAATATGTAGTAAACTCTC |
| | | ATTTATTTGGGGTTCGCTCTTGGATCTCATCCATCC |
| | | ATTGTGTTCTCTTTAATGCTGCCTGCCTTTTGAGGC |
| | | ATTCACTGCCCTAGACAATGCCACCAGAGATAGTG |
| | | GGGGAAATGCCAGATGAAACCAACTCTTGCTCTCA |
| | | CTAGTTGTCAGCTTCTCTGGATAAGTGACCACAGA |
| | | AGCAGGAGTCCTCCTGCTTGGGCATCATTGGGCCA |
| | | GTTCCTTCTCTTTAAATCAGATTTGTAATGGCTCCC |
| | | AAATTCCATCACATCACATTTAAATTGCAGACAGT |
| | | GTTTTGCACATCATGTATCTGTTTTGTCCCATAATA |
| | | TGCTTTTTACTCCCTGATCCCAGTTTCTGCTGTTGAC |
| | | TCTTCCATTCAGTTTTATTTATTGTGTGTTCTCACAG |
| | | TGACACCATTTGTCCTTTTCTGCAACAACCTTTCCA |
| | | GCTACTTTTGCCAAATTCTATTTGTCTTCTCCTTCAA |
| | | AACATTCTCCTTTGCAGTTCCTCTTCATCTGTGTAG |
| | | CTGCTCTTTTGTCTCTTAACTTACCATTCCTATAGTA |
| | | CTTTATGCATCTCTGCTTAGTTCTATTAGTTTTTTGG |
| | | CCTTGCTCTTCTCCTTGATTTTAAAATTCCTTCTATA |
| | | GCTAGAGCTTTTCTTTCTTTCATTCTCTCTTCCTGCA |
| | | GTGTTTTGCATACATCAGAAGCTAGGTACATAAGT |
| | | TAAATGATTGAGAGTTGGCTGTATTTAGATTTATCA |
| | | CTTTTTAATAGGGTGAGCTTGAGAGTTTTCTTTCTT |
| | | TCTGTTTTTTTTTTTTGTTTTTTTTTTTTTTTTTTTTTT |
| | | TTTTTTTTTTGACTAATTTCACATGCTCTAAAAACCT |
| | | TCAAAGGTGATTATTTTTCTCCTGGAAACTCCAGGT |
| | | CCATTCTGTTTAAATCCCTAAGAATGTCAGAATTAA |
| | | AATAACAGGGCTATCCCGTAATTGGAAATATTTCTT |
| | | TTTTCAGGATGCTATAGTCAATTTAGTAAGTGACCA |
| | | CCAAATTGTTATTTGCACTAACAAAGCTCAAAACA |
| | | CGATAAGTTTACTCCTCCATCTCAGTAATAAAAATT |
| | | AAGCTGTAATCAACCTTCTAGGTTTCTCTTGTCTTA |
| | | AAATGGGTATTCAAAAATGGGGATCTGTGGTGTAT |
| | | GTATGGAAACACATACTCCTTAATTTACCTGTTGTT |
| | | GGAAACTGGAGAAATGATTGTCGGGCAACCGTTTA |
| | | TTTTTTATTGTATTTTATTTGGTTGAGGGATTTTTTT |
| | | ATAAACAGTTTTACTTGTGTCATATTTTAAAATTAC |
| | | TAACTGCCATCACCTGCTGGGGTCCTTTGTTAGGTC |
| | | ATTTTCAGTGACTAATAGGGATAATCCAGGTAACT |
| | | TTGAAGAGATGAGCAGTGAGTGACCAGGCAGTTTT |
| | | TCTGCCTTTAGCTTTGACAGTTCTTAATTAAGATCA |
| | | TTGAAGACCAGCTTTCTCATAAATTTCTCTTTTTGA |
| | | AAAAAAGAAAGCATTTGTACTAAGCTCCTCTGTAA |
| | | GACAACATCTTAAATCTTAAAAGTGTTGTTATCATG |
| | | ACTGGTGAGAGAAGAAAACATTTTGTTTTTATTAA |
| | | ATGGAGCATTATTTACAAAAAGCCATTGTTGAGAA |
| | | TTAGATCCCACATCGTATAAATATCTATTAACCATT |
| | | CTAAATAAAGAGAACTCCAGTGTTGCTATGTGCAA |
| | | GATCCTCTCTTGGAGCTTTTTTGCATAGCAATTAAA |
| | | GGTGTGCTATTTGTCAGTAGCCATTTTTTTGCAGTG |
| | | ATTTGAAGACCAAAGTTGTTTTACAGCTGTGTTACC |
| | | GTTAAAGGTTTTTTTTTTTATATGTATTAAATCAATT |
| | | TATCACTGTTTAAAGCTTTGAATATCTGCAATCTTT |
| | | GCCAAGGTACTTTTTTATTTAAAAAAAAACATAAC |
| | | TTTGTAAATATTACCCTGTAATATTATATATACTTA |
| | | ATAAAACATTTTAAGCTATTTTGTTGGGCTATTTCT |
| | | ATTGCTGCTACAGCAGACCACAAGCACATTTCTGA |
| | | AAAATTTAATTTATTAATGTATTTTTAAGTTGCTTA |
| | | TATTCTAGGTAACAATGTAAAGAATGATTTAAAAT |
| | | ATTAATTATGAATTTTTTGAGTATAATACCCAATAA |
| | | GCTTTTAATTAGAGCAGAGTTTTAATTAAAAGTTTT |
| | | AAATCAGTC |
| PREDICTED: Homo | 7 | GTGCGCGCGGCCGTGCAGCCTGGGCAGTGGGTCCT |
| sapiens CD47 molecule | | GCCTGTGACGCGCGGCGGCGGTCGGTCCTGCCTGT |
| (CD47), transcript | | AACGGCGGCGGCGGCTGCTGCTCCGGACACCTGCG |
| variant X11 | | GCGGCGGCGGCGACCCCGCGGCGGGCGCGGAGAT |
| NCBI Reference | | GTGGCCCCTGGTAGCGGCGCTGTTGCTGGGCTCGG |
| Sequence: | | CGTGCTGCGGATCAGCTCAGCTACTATTTAATAAA |
| XM_005247909.2 | | ACAAAATCTGTAGAATTCACGTTTTGTAATGACACT |
| | | GTCGTCATTCCATGCTTTGTTACTAATATGGAGGCA |
| | | CAAAACACTACTGAAGTATACGTAAAGTGGAAATT |
| | | TAAAGGAAGAGATATTTACACCTTTGATGGAGCTC |
| | | TAAACAAGTCCACTGTCCCCACTGACTTTAGTAGTG |
| | | CAAAAATTGAAGTCTCACAATTACTAAAAGGAGAT |
| | | GCCTCTTTGAAGATGGATAAGAGTGATGCTGTCTC |
| | | ACACACAGGAAACTACACTTGTGAAGTAACAGAAT |
| | | TAACCAGAGAAGGTGAAACGATCATCGAGCTAAA |
| | | ATATCGTGTTGTTTCATGGTTTTCTCCAAATGAAAA |
| | | TATTCTTATTGTTATTTTCCCAATTTTTGCTATACTC |
| | | CTGTTCTGGGGACAGTTTGGTATTAAAACACTTAA |
| | | ATATAGATCCGGTGGTATGGATGAGAAAACAATTG |
| | | CTTTACTTGTTGCTGGACTAGTGATCACTGTCATTG |
| | | TCATTGTTGGAGCCATTCTTTTCGTCCCAGGTGAAT |
| | | ATTCATTAAAGAATGCTACTGGCCTTGGTTTAATTG |
| | | TGACTTCTACAGGGATATTAATATTACTTCACTACT |
| | | ATGTGTTTAGTACAGCGATTGGATTAACCTCCTTCG |
| | | TCATTGCCATATTGGTTATTCAGGTGATAGCCTATA |
| | | TCCTCGCTGTGGTTGGACTGAGTCTCTGTATTGCGG |
| | | CGTGTATACCAATGCATGGCCCTCTTCTGATTTCAG |
| | | GTTTGAGTATCTTAGCTCTAGCACAATTACTTGGAC |
| | | TAGTTTATATGAAATTTGTGGAATAACTGAAGTGA |
| | | AGTGATGGACTCCGATTTGGAGAGTAGTAAGACGT |
| | | GAAAGGAATACACTTGTGTTTAAGCACCATGGCCT |
| | | TGATGATTCACTGTTGGGGAGAAGAAACAAGAAAA |
| | | GTAACTGGTTGTCACCTATGAGACCCTTACGTGATT |
| | | GTTAGTTAAGTTTTTATTCAAAGCAGCTGTAATTTA |
| | | GTTAATAAAATAATTATGATCTATGTTGTTTGCCCA |
| | | ATTGAGATCCAGTTTTTTGTTGTTATTTTTAATCAAT |
| | | TAGGGGCAATAGTAGAATGGACAATTTCCAAGAAT |
| | | GATGCCTTTCAGGTCCTAGGGCCTCTGGCCTCTAGG |
| | | TAACCAGTTTAAATTGGTTCAGGGTGATAACTACTT |
| | | AGCACTGCCCTGGTGATTACCCAGAGATATCTATG |
| | | AAAACCAGTGGCTTCCATCAAACCTTTGCCAACTC |
| | | AGGTTCACAGCAGCTTTGGGCAGTTATGGCAGTAT |
| | | GGCATTAGCTGAGAGGTGTCTGCCACTTCTGGGTC |
| | | AATGGAATAATAAATTAAGTACAGGCAGGAATTTG |
| | | GTTGGGAGCATCTTGTATGATCTCCGTATGATGTGA |
| | | TATTGATGGAGATAGTGGTCCTCATTCTTGGGGGTT |
| | | GCCATTCCCACATTCCCCCTTCAACAAACAGTGTAA |
| | | CAGGTCCTTCCCAGATTTAGGGTACTTTTATTGATG |
| | | GATATGTTTTCCTTTTATTCACATAACCCCTTGAAA |
| | | CCCTGTCTTGTCCTCCTGTTACTTGCTTCTGCTGTAC |
| | | AAGATGTAGCACCTTTTCTCCTCTTTGAACATGGTC |
| | | TAGTGACACGGTAGCACCAGTTGCAGGAAGGAGCC |
| | | AGACTTGTTCTCAGAGCACTGTGTTCACACTTTTCA |
| | | GCAAAAATAGCTATGGTTGTAACATATGTATTCCCT |
| | | TCCTCTGATTTGAAGGCAAAAATCTACAGTGTTTCT |
| | | TCACTTCTTTTCTGATCTGGGGCATGAAAAAAGCA |
| | | AGATTGAAATTTGAACTATGAGTCTCCTGCATGGC |
| | | AACAAAATGTGTGTCACCATCAGGCCAACAGGCCA |
| | | GCCCTTGAATGGGGATTTATTACTGTTGTATCTATG |
| | | TTGCATGATAAACATTCATCACCTTCCTCCTGTAGT |
| | | CCTGCCTCGTACTCCCCTTCCCCTATGATTGAAAAG |
| | | TAAACAAAACCCACATTTCCTATCCTGGTTAGAAG |
| | | AAAATTAATGTTCTGACAGTTGTGATCGCCTGGAG |
| | | TACTTTTAGACTTTTAGCATTCGTTTTTTACCTGTTT |
| | | GTGGATGTGTGTTTGTATGTGCATACGTATGAGATA |
| | | GGCACATGCATCTTCTGTATGGACAAAGGTGGGGT |
| | | ACCTACAGGAGAGCAAAGGTTAATTTTGTGCTTTT |
| | | AGTAAAAACATTTAAATACAAAGTTCTTTATTGGG |
| | | TGGAATTATATTTGATGCAAATATTTGATCACTTAA |
| | | AACTTTTAAAACTTCTAGGTAATTTGCCACGCTTTT |
| | | TGACTGCTCACCAATACCCTGTAAAAATACGTAAT |
| | | TCTTCCTGTTTGTGTAATAAGATATTCATATTTGTA |
| | | GTTGCATTAATAATAGTTATTTCTTAGTCCATCAGA |
| | | TGTTCCCGTGTGCCTCTTTTATGCCAAATTGATTGT |
| | | CATATTTCATGTTGGGACCAAGTAGTTTGCCCATGG |
| | | CAAACCTAAATTTATGACCTGCTGAGGCCTCTCAG |
| | | AAAACTGAGCATACTAGCAAGACAGCTCTTCTTGA |
| | | AAAAAAAAATATGTATACACAAATATATACGTATA |
| | | TCTATATATACGTATGTATATACACACATGTATATT |
| | | CTTCCTTGATTGTGTAGCTGTCCAAAATAATAACAT |
| | | ATATAGAGGGAGCTGTATTCCTTTATACAAATCTG |
| | | ATGGCTCCTGCAGCACTTTTTCCTTCTGAAAATATT |
| | | TACATTTTGCTAACCTAGTTTGTTACTTTAAAAATC |
| | | AGTTTTGATGAAAGGAGGGAAAAGCAGATGGACTT |
| | | GAAAAAGATCCAAGCTCCTATTAGAAAAGGTATGA |
| | | AAATCTTTATAGTAAAATTTTTTATAAACTAAAGTT |
| | | GTACCTTTTAATATGTAGTAAACTCTCATTTATTTG |
| | | GGGTTCGCTCTTGGATCTCATCCATCCATTGTGTTC |
| | | TCTTTAATGCTGCCTGCCTTTTGAGGCATTCACTGC |
| | | CCTAGACAATGCCACCAGAGATAGTGGGGGAAATG |
| | | CCAGATGAAACCAACTCTTGCTCTCACTAGTTGTCA |
| | | GCTTCTCTGGATAAGTGACCACAGAAGCAGGAGTC |
| | | CTCCTGCTTGGGCATCATTGGGCCAGTTCCTTCTCT |
| | | TTAAATCAGATTTGTAATGGCTCCCAAATTCCATCA |
| | | CATCACATTTAAATTGCAGACAGTGTTTTGCACATC |
| | | ATGTATCTGTTTTGTCCCATAATATGCTTTTTACTCC |
| | | CTGATCCCAGTTTCTGCTGTTGACTCTTCCATTCAG |
| | | TTTTATTTATTGTGTGTTCTCACAGTGACACCATTT |
| | | GTCCTTTTCTGCAACAACCTTTCCAGCTACTTTTGC |
| | | CAAATTCTATTTGTCTTCTCCTTCAAAACATTCTCCT |
| | | TTGCAGTTCCTCTTCATCTGTGTAGCTGCTCTTTTGT |
| | | CTCTTAACTTACCATTCCTATAGTACTTTATGCATC |
| | | TCTGCTTAGTTCTATTAGTTTTTTGGCCTTGCTCTTC |
| | | TCCTTGATTTTAAAATTCCTTCTATAGCTAGAGCTT |
| | | TTCTTTCTTTCATTCTCTCTTCCTGCAGTGTTTTGCA |
| | | TACATCAGAAGCTAGGTACATAAGTTAAATGATTG |
| | | AGAGTTGGCTGTATTTAGATTTATCACTTTTTAATA |
| | | GGGTGAGCTTGAGAGTTTTCTTTCTTTCTGTTTTTTT |
| | | TTTTTGTTTTTTTTTTTTTTTTTTTTTTTTTTTTTTTTG |
| | | ACTAATTTCACATGCTCTAAAAACCTTCAAAGGTG |
| | | ATTATTTTTCTCCTGGAAACTCCAGGTCCATTCTGT |
| | | TTAAATCCCTAAGAATGTCAGAATTAAAATAACAG |
| | | GGCTATCCCGTAATTGGAAATATTTCTTTTTTCAGG |
| | | ATGCTATAGTCAATTTAGTAAGTGACCACCAAATT |
| | | GTTATTTGCACTAACAAAGCTCAAAACACGATAAG |
| | | TTTACTCCTCCATCTCAGTAATAAAAATTAAGCTGT |
| | | AATCAACCTTCTAGGTTTCTCTTGTCTTAAAATGGG |
| | | TATTCAAAAATGGGGATCTGTGGTGTATGTATGGA |
| | | AACACATACTCCTTAATTTACCTGTTGTTGGAAACT |
| | | GGAGAAATGATTGTCGGGCAACCGTTTATTTTTTAT |
| | | TGTATTTTATTTGGTTGAGGGATTTTTTTATAAACA |
| | | GTTTTACTTGTGTCATATTTTAAAATTACTAACTGC |
| | | CATCACCTGCTGGGGTCCTTTGTTAGGTCATTTTCA |
| | | GTGACTAATAGGGATAATCCAGGTAACTTTGAAGA |
| | | GATGAGCAGTGAGTGACCAGGCAGTTTTTCTGCCT |
| | | TTAGCTTTGACAGTTCTTAATTAAGATCATTGAAGA |
| | | CCAGCTTTCTCATAAATTTCTCTTTTTGAAAAAAAG |
| | | AAAGCATTTGTACTAAGCTCCTCTGTAAGACAACA |
| | | TCTTAAATCTTAAAAGTGTTGTTATCATGACTGGTG |
| | | AGAGAAGAAAACATTTTGTTTTTATTAAATGGAGC |
| | | ATTATTTACAAAAAGCCATTGTTGAGAATTAGATC |
| | | CCACATCGTATAAATATCTATTAACCATTCTAAATA |
| | | AAGAGAACTCCAGTGTTGCTATGTGCAAGATCCTC |
| | | TCTTGGAGCTTTTTTGCATAGCAATTAAAGGTGTGC |
| | | TATTTGTCAGTAGCCATTTTTTTGCAGTGATTTGAA |
| | | GACCAAAGTTGTTTTACAGCTGTGTTACCGTTAAAG |
| | | GTTTTTTTTTTTATATGTATTAAATCAATTTATCACT |
| | | GTTTAAAGCTTTGAATATCTGCAATCTTTGCCAAGG |
| | | TACTTTTTTATTTAAAAAAAAACATAACTTTGTAAA |
| | | TATTACCCTGTAATATTATATATACTTAATAAAACA |
| | | TTTTAAGCTA |
| Homo sapiens CD47 | 8 | GCAGCCTGGGCAGTGGGTCCTGCCTGTGACGCGCG |
| molecule (CD47), | | GCGGCGGTCGGTCCTGCCTGTAACGGCGGCGGCGG |
| transcript variant 3 | | CTGCTGCTCCGGACACCTGCGGCGGCGGCGGCGAC |
| NCBI Reference | | CCCGCGGCGGGCGCGGAGATGTGGCCCCTGGTAGC |
| Sequence: | | GGCGCTGTTGCTGGGCTCGGCGTGCTGCGGATCAG |
| NM_001382306.1 | | CTCAGCTACTATTTAATAAAACAAAATCTGTAGAA |
| | | TTCACGTTTTGTAATGACACTGTCGTCATTCCATGC |
| | | TTTGTTACTAATATGGAGGCACAAAACACTACTGA |
| | | AGTATACGTAAAGTGGAAATTTAAAGGAAGAGATA |
| | | TTTACACCTTTGATGGAGCTCTAAACAAGTCCACTG |
| | | TCCCCACTGACTTTAGTAGTGCAAAAATTGAAGTCT |
| | | CACAATTACTAAAAGGAGATGCCTCTTTGAAGATG |
| | | GATAAGAGTGATGCTGTCTCACACACAGGAAACTA |
| | | CACTTGTGAAGTAACAGAATTAACCAGAGAAGGTG |
| | | AAACGATCATCGAGCTAAAATATCGTGTTGTTTCAT |
| | | GGTTTTCTCCAAATGAAAATATTCTTATTGTTATTT |
| | | TCCCAATTTTTGCTATACTCCTGTTCTGGGGACAGT |
| | | TTGGTATTAAAACACTTAAATATAGATCCGGTGGT |
| | | ATGGATGAGAAAACAATTGCTTTACTTGTTGCTGG |
| | | ACTAGTGATCACTGTCATTGTCATTGTTGGAGCCAT |
| | | TCTTTTCGTCCCAGGTGAATATTCATTAAAGAATGC |
| | | TACTGGCCTTGGTTTAATTGTGACTTCTACAGGGAT |
| | | ATTAATATTACTTCACTACTATGTGTTTAGTACAGC |
| | | GATTGGATTAACCTCCTTCGTCATTGCCATATTGGT |
| | | TATTCAGGTGATAGCCTATATCCTCGCTGTGGTTGG |
| | | ACTGAGTCTCTGTATTGCGGCGTGTATACCAATGCA |
| | | TGGCCCTCTTCTGATTTCAGGTTTGAGTATCTTAGC |
| | | TCTAGCACAATTACTTGGACTAGTTTATATGAAATT |
| | | TGTGGCTTCCAATCAGAAGACTATACAACCTCCTA |
| | | GGAAAGCTGTAGAGGAACCCCTTAATGAATAACTG |
| | | AAGTGAAGTGATGGACTCCGATTTGGAGAGTAGTA |
| | | AGACGTGAAAGGAATACACTTGTGTTTAAGCACCA |
| | | TGGCCTTGATGATTCACTGTTGGGGAGAAGAAACA |
| | | AGAAAAGTAACTGGTTGTCACCTATGAGACCCTTA |
| | | CGTGATTGTTAGTTAAGTTTTTATTCAAAGCAGCTG |
| | | TAATTTAGTTAATAAAATAATTATGATCTATGTTGT |
| | | TTGCCCAATTGAGATCCAGTTTTTTGTTGTTATTTTT |
| | | AATCAATTAGGGGCAATAGTAGAATGGACAATTTC |
| | | CAAGAATGATGCCTTTCAGGTCCTAGGGCCTCTGG |
| | | CCTCTAGGTAACCAGTTTAAATTGGTTCAGGGTGAT |
| | | AACTACTTAGCACTGCCCTGGTGATTACCCAGAGA |
| | | TATCTATGAAAACCAGTGGCTTCCATCAAACCTTTG |
| | | CCAACTCAGGTTCACAGCAGCTTTGGGCAGTTATG |
| | | GCAGTATGGCATTAGCTGAGAGGTGTCTGCCACTT |
| | | CTGGGTCAATGGAATAATAAATTAAGTACAGGCAG |
| | | GAATTTGGTTGGGAGCATCTTGTATGATCTCCGTAT |
| | | GATGTGATATTGATGGAGATAGTGGTCCTCATTCTT |
| | | GGGGGTTGCCATTCCCACATTCCCCCTTCAACAAAC |
| | | AGTGTAACAGGTCCTTCCCAGATTTAGGGTACTTTT |
| | | ATTGATGGATATGTTTTCCTTTTATTCACATAACCC |
| | | CTTGAAACCCTGTCTTGTCCTCCTGTTACTTGCTTCT |
| | | GCTGTACAAGATGTAGCACCTTTTCTCCTCTTTGAA |
| | | CATGGTCTAGTGACACGGTAGCACCAGTTGCAGGA |
| | | AGGAGCCAGACTTGTTCTCAGAGCACTGTGTTCAC |
| | | ACTTTTCAGCAAAAATAGCTATGGTTGTAACATAT |
| | | GTATTCCCTTCCTCTGATTTGAAGGCAAAAATCTAC |
| | | AGTGTTTCTTCACTTCTTTTCTGATCTGGGGCATGA |
| | | AAAAAGCAAGATTGAAATTTGAACTATGAGTCTCC |
| | | TGCATGGCAACAAAATGTGTGTCACCATCAGGCCA |
| | | ACAGGCCAGCCCTTGAATGGGGATTTATTACTGTT |
| | | GTATCTATGTTGCATGATAAACATTCATCACCTTCC |
| | | TCCTGTAGTCCTGCCTCGTACTCCCCTTCCCCTATG |
| | | ATTGAAAAGTAAACAAAACCCACATTTCCTATCCT |
| | | GGTTAGAAGAAAATTAATGTTCTGACAGTIGTGAT |
| | | CGCCTGGAGTACTTTTAGACTTTTAGCATTCGTTTT |
| | | TTACCTGTTTGTGGATGTGTGTTTGTATGTGCATAC |
| | | GTATGAGATAGGCACATGCATCTTCTGTATGGACA |
| | | AAGGTGGGGTACCTACAGGAGAGCAAAGGTTAATT |
| | | TTGTGCTTTTAGTAAAAACATTTAAATACAAAGTTC |
| | | TTTATTGGGTGGAATTATATTTGATGCAAATATTTG |
| | | ATCACTTAAAACTTTTAAAACTTCTAGGTAATTTGC |
| | | CACGCTTTTTGACTGCTCACCAATACCCTGTAAAAA |
| | | TACGTAATTCTTCCTGTTTGTGTAATAAGATATTCA |
| | | TATTTGTAGTTGCATTAATAATAGTTATTTCTTAGT |
| | | CCATCAGATGTTCCCGTGTGCCTCTTTTATGCCAAA |
| | | TTGATTGTCATATTTCATGTTGGGACCAAGTAGTTT |
| | | GCCCATGGCAAACCTAAATTTATGACCTGCTGAGG |
| | | CCTCTCAGAAAACTGAGCATACTAGCAAGACAGCT |
| | | CTTCTTGAAAAAAAAAATATGTATACACAAATATA |
| | | TACGTATATCTATATATACGTATGTATATACACACA |
| | | TGTATATTCTTCCTTGATTGTGTAGCTGTCCAAAAT |
| | | AATAACATATATAGAGGGAGCTGTATTCCTTTATA |
| | | CAAATCTGATGGCTCCTGCAGCACTTTTTCCTTCTG |
| | | AAAATATTTACATTTTGCTAACCTAGTTTGTTACTT |
| | | TAAAAATCAGTTTTGATGAAAGGAGGGAAAAGCA |
| | | GATGGACTTGAAAAAGATCCAAGCTCCTATTAGAA |
| | | AAGGTATGAAAATCTTTATAGTAAAATTTTTTATAA |
| | | ACTAAAGTTGTACCTTTTAATATGTAGTAAACTCTC |
| | | ATTTATTTGGGGTTCGCTCTTGGATCTCATCCATCC |
| | | ATTGTGTTCTCTTTAATGCTGCCTGCCTTTTGAGGC |
| | | ATTCACTGCCCTAGACAATGCCACCAGAGATAGTG |
| | | GGGGAAATGCCAGATGAAACCAACTCTTGCTCTCA |
| | | CTAGTTGTCAGCTTCTCTGGATAAGTGACCACAGA |
| | | AGCAGGAGTCCTCCTGCTTGGGCATCATTGGGCCA |
| | | GTTCCTTCTCTTTAAATCAGATTTGTAATGGCTCCC |
| | | AAATTCCATCACATCACATTTAAATTGCAGACAGT |
| | | GTTTTGCACATCATGTATCTGTTTTGTCCCATAATA |
| | | TGCTTTTTACTCCCTGATCCCAGTTTCTGCTGTTGAC |
| | | TCTTCCATTCAGTTTTATTTATTGTGTGTTCTCACAG |
| | | TGACACCATTTGTCCTTTTCTGCAACAACCTTTCCA |
| | | GCTACTTTTGCCAAATTCTATTTGTCTTCTCCTTCAA |
| | | AACATTCTCCTTTGCAGTTCCTCTTCATCTGTGTAG |
| | | CTGCTCTTTTGTCTCTTAACTTACCATTCCTATAGTA |
| | | CTTTATGCATCTCTGCTTAGTTCTATTAGTTTTTTGG |
| | | CCTTGCTCTTCTCCTTGATTTTAAAATTCCTTCTATA |
| | | GCTAGAGCTTTTCTTTCTTTCATTCTCTCTTCCTGCA |
| | | GTGTTTTGCATACATCAGAAGCTAGGTACATAAGT |
| | | TAAATGATTGAGAGTTGGCTGTATTTAGATTTATCA |
| | | CTTTTTAATAGGGTGAGCTTGAGAGTTTTCTTTCTT |
| | | TCTGTTTTTTTTTTTTGTTTTTTTTTTTTTTTTTTTTTT |
| | | TTTTTTTTTTGACTAATTTCACATGCTCTAAAAACCT |
| | | TCAAAGGTGATTATTTTTCTCCTGGAAACTCCAGGT |
| | | CCATTCTGTTTAAATCCCTAAGAATGTCAGAATTAA |
| | | AATAACAGGGCTATCCCGTAATTGGAAATATTTCTT |
| | | TTTTCAGGATGCTATAGTCAATTTAGTAAGTGACCA |
| | | CCAAATTGTTATTTGCACTAACAAAGCTCAAAACA |
| | | CGATAAGTTTACTCCTCCATCTCAGTAATAAAAATT |
| | | AAGCTGTAATCAACCTTCTAGGTTTCTCTTGTCTTA |
| | | AAATGGGTATTCAAAAATGGGGATCTGTGGTGTAT |
| | | GTATGGAAACACATACTCCTTAATTTACCTGTTGTT |
| | | GGAAACTGGAGAAATGATTGTCGGGCAACCGTTTA |
| | | TTTTTTATTGTATTTTATTTGGTTGAGGGATTTTTTT |
| | | ATAAACAGTTTTACTTGTGTCATATTTTAAAATTAC |
| | | TAACTGCCATCACCTGCTGGGGTCCTTTGTTAGGTC |
| | | ATTTTCAGTGACTAATAGGGATAATCCAGGTAACT |
| | | TTGAAGAGATGAGCAGTGAGTGACCAGGCAGTTTT |
| | | TCTGCCTTTAGCTTTGACAGTTCTTAATTAAGATCA |
| | | TTGAAGACCAGCTTTCTCATAAATTTCTCTTTTTGA |
| | | AAAAAAGAAAGCATTTGTACTAAGCTCCTCTGTAA |
| | | GACAACATCTTAAATCTTAAAAGTGTTGTTATCATG |
| | | ACTGGTGAGAGAAGAAAACATTTTGTTTTTATTAA |
| | | ATGGAGCATTATTTACAAAAAGCCATTGTTGAGAA |
| | | TTAGATCCCACATCGTATAAATATCTATTAACCATT |
| | | CTAAATAAAGAGAACTCCAGTGTTGCTATGTGCAA |
| | | GATCCTCTCTTGGAGCTTTTTTGCATAGCAATTAAA |
| | | GGTGTGCTATTTGTCAGTAGCCATTTTTTTGCAGTG |
| | | ATTTGAAGACCAAAGTTGTTTTACAGCTGTGTTACC |
| | | GTTAAAGGTTTTTTTTTTTATATGTATTAAATCAATT |
| | | TATCACTGTTTAAAGCTTTGAATATCTGCAATCTTT |
| | | GCCAAGGTACTTTTTTATTTAAAAAAAAACATAAC |
| | | TTTGTAAATATTACCCTGTAATATTATATATACTTA |
| | | ATAAAACATTTTAAGCTATTTTGTTGGGCTATTTCT |
| | | ATTGCTGCTACAGCAGACCACAAGCACATTTCTGA |
| | | AAAATTTAATTTATTAATGTATTTTTAAGTTGCTTA |
| | | TATTCTAGGTAACAATGTAAAGAATGATTTAAAAT |
| | | ATTAATTATGAATTTTTTGAGTATAATACCCAATAA |
| | | GCTTTTAATTAGAGCAGAGTTTTAATTAAAAGTTTT |
| | | AAATCAGTCCAA |
| leukocyte surface | 9 | MWPLAAALLLGSCCCGSAQLLFSNVNSIEFTSCNETV |
| antigen CD47 isoform | | VIPCIVRNVEAQSTEEMFVKWKLNKSYIFIYDGNKNS |
| 4 precursor [Mus | | TTTDQNFTSAKISVSDLINGIASLKMDKRDAMVGNYT |
| musculus] | | CEVTELSREGKTVIELKNRTAFNTDQGSACSYEEEKG |
| NCBI Reference | | GCKLVSWFSPNEKILIVIFPILAILLFWGKFGILTLKYK |
| Sequence: | | SSHTNKRIILLLVAGLVLTVIVVVGAILLIPGEKPVKN |
| NP_034711.1 | | ASGLGLIVISTGILILLQYNVFMTAFGMTSFTIAILITQV |
| | | LGYVLALVGLCLCIMACEPVHGPLLISGLGIIALAELL |
| | | GLVYMKFVASNQRTIQPPRNR |

### EXAMPLES

The examples in this section (i.e., section 7) are offered by way of illustration, and not by way of limitation.

### 9.1 Example 1: Cross-Dressing of Pig LCL and Human Jurkat Cells by Transgenic hCD47 after Co-Culture with hCD47-Tg LCL Cells

This example demonstrates that heterologous human CD47 can be successfully expressed in a non-human CD47 expressing cell line after co-culture.

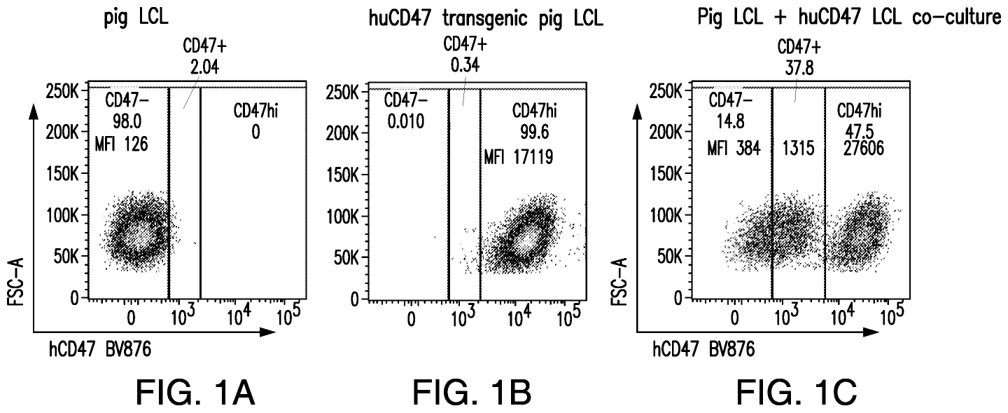

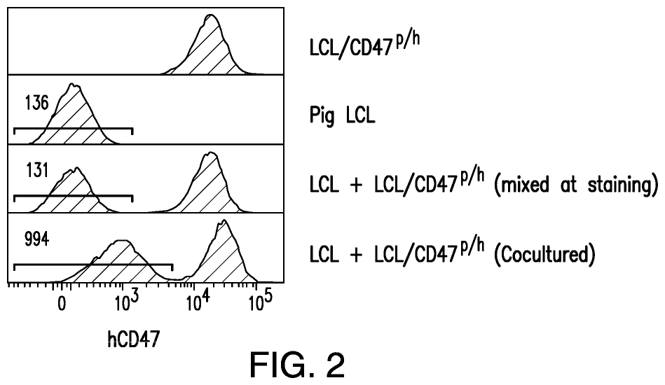

To determine whether human CD47 could be transferred from one cell to another by EVs, cells from a parental pig B-lymphoma cell line (LCL) that did not express human CD47 (hCD47) were co-cultured with LCL cells that transgenically express hCD47. As shown in FIG. 1A and FIG. 2, the parental pig LCL cell line did not express hCD47, as measured by FACS (FIG. 1A, FIG. 2, and Table 2). In contrast, the pig LCL cells expressing transgenic human CD47 (hCD47-Tg LCL cells) expressed high levels of CD47 (FIG. 1B, FIG. 2, and Table 2). Notably, detection of huCD47 in the parental LCL cell line following co-culture with hCD47-Tg LCL cells indicated a strong increase in hCD47 expression (FIG. 1C, FIG. 2, and Table 2).

TABLE 2
FACS results of hCD47 expression in LCL cells

| | hCD47 negative (%) | hCD47+ (%) | High hCD47+ (%) |
|---|---|---|---|
| LCL cells | 98 | 2.04 | 0 |
| hCD47-Tg LCL cells | 0.010 | 0.34 | 99.6 |
| LCL cells + hCD47- | 14.8 | 37.8 | 47.5 |
| Tg LCL cells co- | | | |
| culture | | | |
| CD47 KO Jurkat | 99.5 | 0.49 | 0 |
| cells | | | |
| CD47 KO Jurkat + | 39.7 | 16.8 | 43.4 |
| hCD47-Tg LCL cells | | | |
| co-culture | | | |

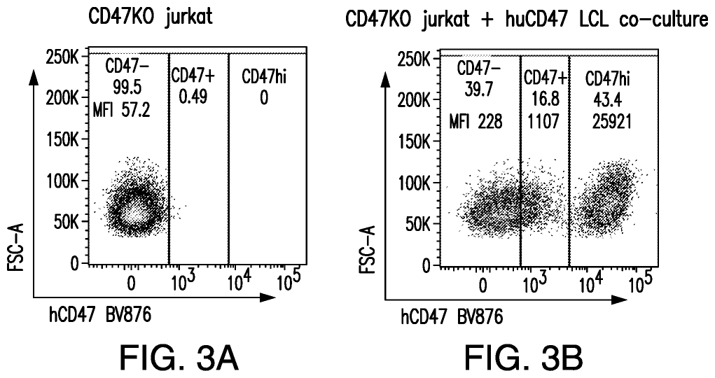

Similar results were observed using human T-cell leukemia Jurkat cells in which CD47 was knocked out by CRISPR-Cas9, and co-cultured with the hCD47-Tg LCL cells. Briefly, CD47 knockout (KO) Jurkat cells were co-cultured with the hCD47-Tg LCL cells and hCD47 expression was measured by FACS. As shown in FIG. 3B, co-culture led to a strong increase in hCD47 expression in the CD47KO Jurkat cells (FIG. 3B, and Table 2).

These data indicated that porcine cells can be cross-dressed with human CD47 by co-culture with cells that express human CD47.

### 9.2 Example 2: Cross-Dressing of Pig LCL and hCD47 Knockout Jurkat Cells by Native hCD47 after Co-Culture with Wildtype Jurkat Cells

This example demonstrates that cross-dressing of hCD47 from one cell line to another following co-culture was reproducible in additional exemplary cell line models.

Briefly, CD47 knockout (KO) cells were co-cultured with the parental (wildtype, WT) Jurkat cells or pig LCL cells for 24 h, and analyzed for hCD47 cross-dressing on gated CD47KO Jurkat cells by FACS using anti-hCD47-BV786 mAb. The cells that were cultured alone were used as staining controls, which were either stained separately or mixed immediately prior to staining. Shown are representative histograms (the numbers in the figure indicate mean fluorescence intensity (MFI) of gated CD47KO Jurkat cells).

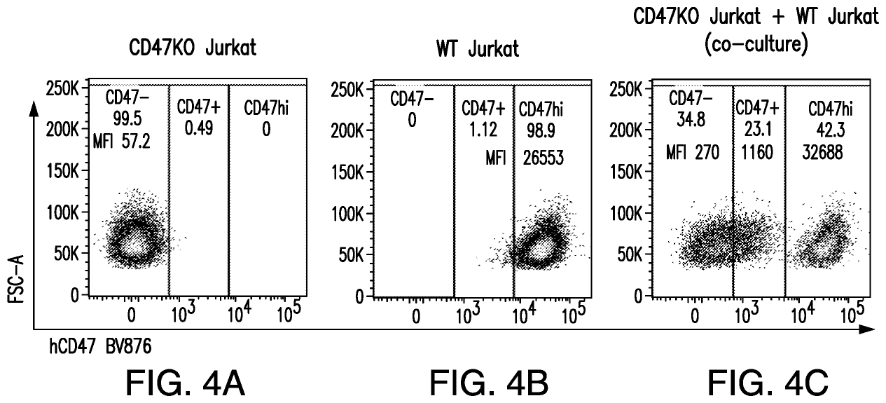

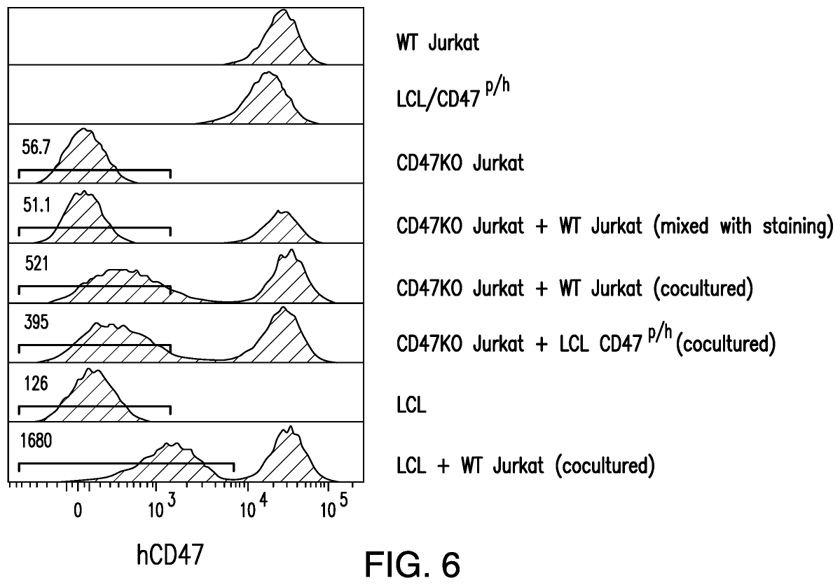

As shown in FIG. 4A, the levels of human CD47 as determined by FACS in CD47KO Jurkat cells was almost completely undetectable, relative to wild-type (WT) Jurkat cells (FIG. 4 and FIG. 4B, respectively; FIG. 6, and Table 3). Following co-culture of CD47KO Jurkat cells with WT Jurkat cells, a strong increase in hCD47 was observed in the CD47KO Jurkat cells (FIG. 4C, FIG. 6, and Table 3).

TABLE 3
FACS results of hCD47 expression in
CD47KO Jurkat cells and pig LCL cells

| | hCD47 | hCD47 | High hCD47 |
|---|---|---|---|
| | negative (%) | positive (%) | positive (%) |
| CD47 KO Jurkat | 99.5 | 0.49 | 0 |
| WT Jurkat | 0 | 1.12 | 98.9 |
| CD47 KO Jurkat + WT Jurkat | 34.8 | 23.1 | 42.3 |
| (co-culture) | | | |
| Pig LCL | 98.0 | 2.04 | 0 |
| Pig LCL + WT Jurkat (co- | 6.82 | 47.4 | 45.7 |
| culture) | | | |

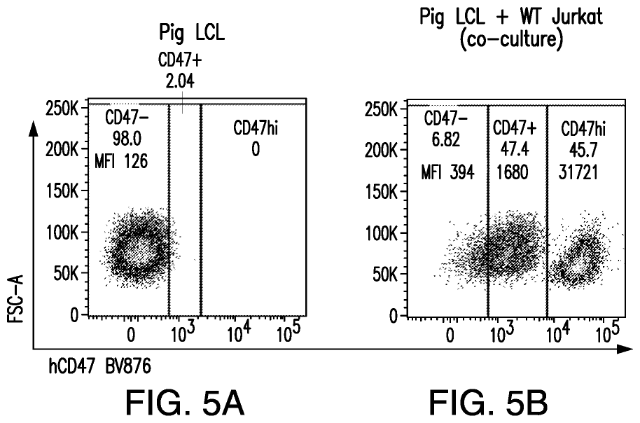

Similar results were observed when Pig LCL cells were co-cultured with WT Jurkat cells. As shown in FIGS. 5A-5B, co-culture of pig LCL cells with WT Jurkat cells resulted in a strong increase in hCD47 expression relative to non-co cultured pig LCL cells (FIG. 5A and FIG. 5B, respectively, and Table 3).

These results indicated that human CD47 cross-dressing can also occur on human cells, and can be induced by not only hCD47-transgenic cells but also cells that only express native CD47.

### 9.3 Example 3: CD47 Cross-Dressing of CD47KO Jurkat Cells by Extracellular Vesicles or Exosomes from WT Jurkat Cells

This example demonstrates that isolated EVs from cells expressing CD47 can be used to cross-dress cells that do not express CD47.

Briefly, MVs and exosomes were isolated from the supernatants collected from WT Jurkat cells cultured in 10% exosome depleted FBS. Extracellular vesicles (EVs) and exosomes (Exos) from cell culture supernatants were purified by a standard differential centrifugation protocol. Supernatants collected from 48 h cell cultures were centrifuged at 2,000 g (3,000 rpm) for 20 min to remove cell debris and dead cells. Extracellular vesicles were pelleted after centrifugation at 16,500 g (9,800 rpm) for 45 min (Beckman Coulter, Optima XE-90) and resuspended in PBS. The pelleted exosomes from the supernatants were further centrifuged at 100,000 g (26,450 rpm) for 2 h at 4° C. (Beckman Coulter, Optima XE-90) and resuspended in PBS.

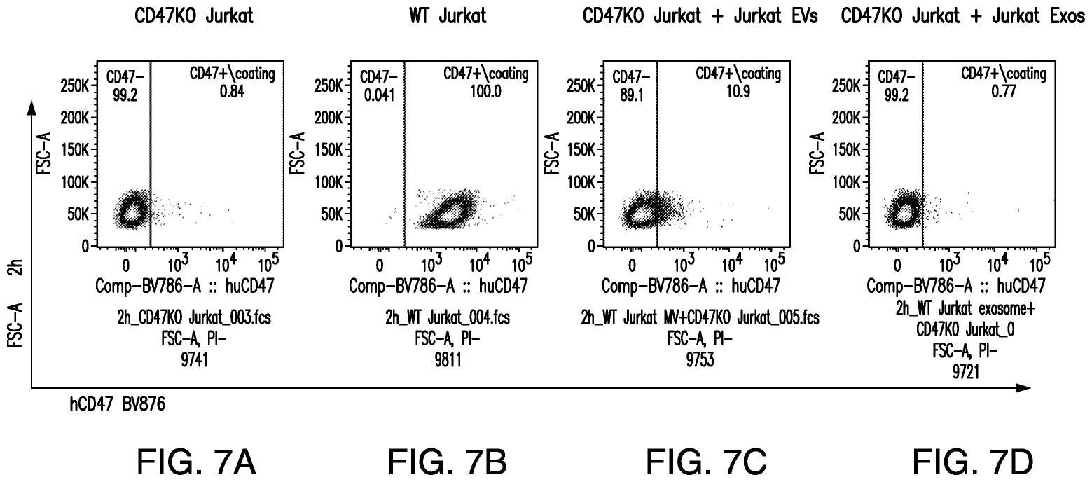

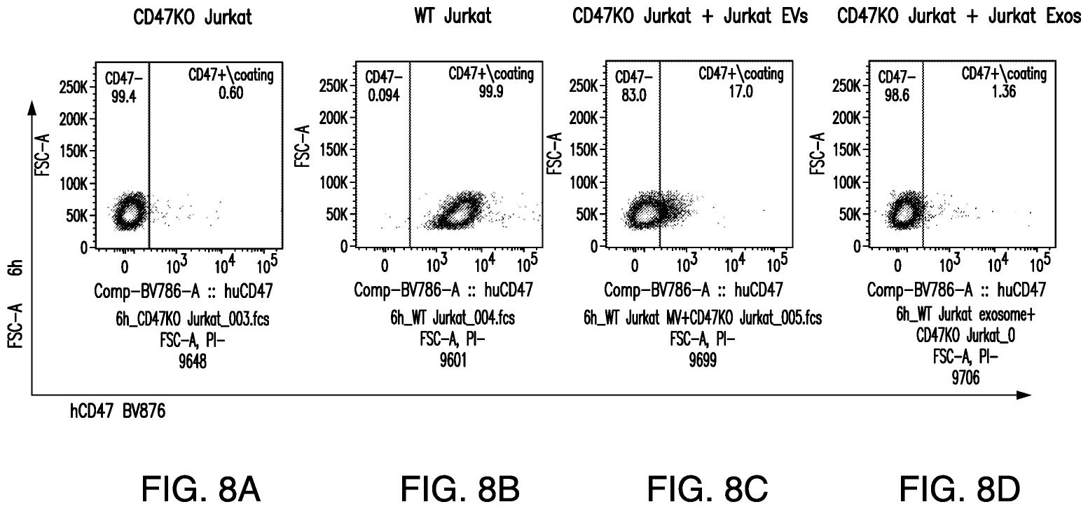

The isolated MVs and exosomes were subsequently co-cultured with Jurkat cells in which CD47 was knocked out (CD47 KO Jurkat cells) for 2 h or 6 h. As shown in FIG. 7C, co-culture for 2 h with MVs led to an increase in CD47 expression in the CD47 KO Jurkat cells (FIG. 7C, and Table 4). After 6 h of co-culture with MV or exosomes, CD47 expression was increased in the CD47 KO Jurkat cells (FIG. 8C and FIG. 8D, respectively, and Table 4).

TABLE 4
FACS results of hCD47 expression in CD47KO Jurkat cells

| | hCD47 negative | hCD47 positive |
|---|---|---|
| | (%) | (%) |
| CD47 KO Jurkat cells | 99.2 | 0.84 |
| WT Jurkat cells | 0.041 | 100 |
| CD47 KO Jurkat cells + Jurkat WT | 89.1 | 10.9 |
| MV (2 hours) | | |
| CD47 KO Jurkat cells + Jurkat WT | 99.2 | 0.77 |
| Exosomes (2 hours) | | |
| CD47 KO Jurkat cells | 99.4 | 0.60 |
| WT Jurkat cells | 0.094 | 99.9 |
| CD47 KO Jurkat cells + Jurkat WT | 83.0 | 17 |
| MV (6 hours) | | |
| CD47 KO Jurkat cells + Jurkat WT | 98.6 | 1.36 |
| Exosomes (6 hours) | | |

These data indicate that EVs (e.g., MVs or exosomes) from cells expressing hCD47 can be used to cross-dress cells that do not express hCD47.

### 9.4 Example 4: CD47 Cross-Dressing of Pig LCL Cells by Extracellular Vesicles or Exosomes from WT Jurkat Cells

This example demonstrates that porcine cells can be cross-dressed with hCD47 from human cells following co-culture.

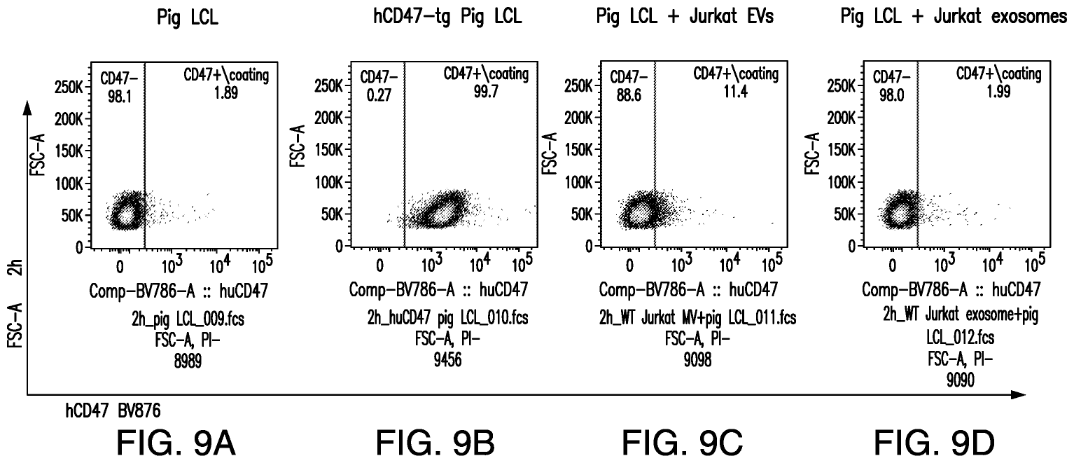

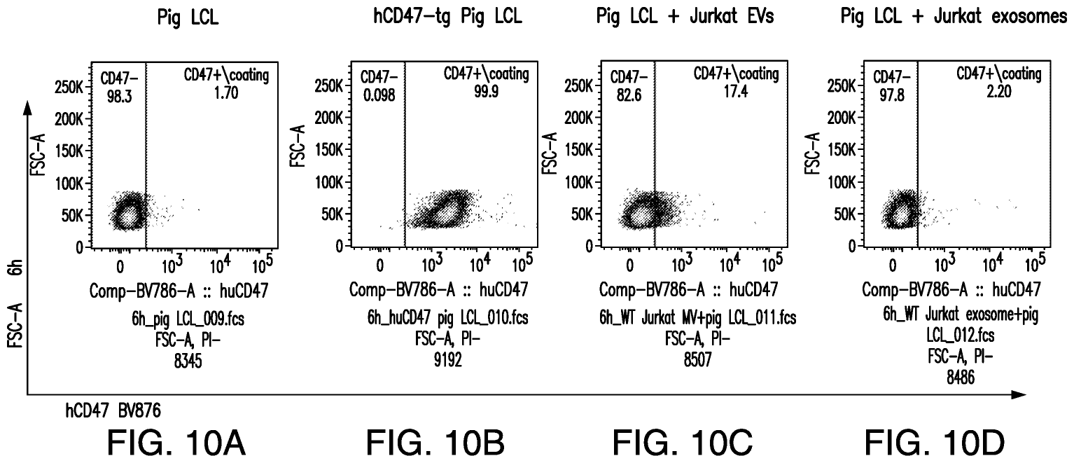

Briefly, MVs and exosomes were isolated from WT Jurkat cells, as described above. The isolated MVs and exosomes were subsequently co-cultured with pig LCL cells that do not express hCD47 for 2 h or 6 h. As shown in FIG. 9C, co-culture for 2 h with MVs led to an increase in CD47 expression in the pig LCL cells (FIG. 9C, and Table 5). After 6 h of co-culture with MV or exosomes, CD47 expression was increased in the pig LCL cells (FIG. 10C and FIG. 10D, respectively, and Table 5).

TABLE 5
FACS results of hCD47 expression in pig LCL cells

| | hCD47 negative | hCD47 positive |
|---|---|---|
| | (%) | (%) |
| Pig LCL cells | 98.1 | 1.89 |
| hCD47-Tg LCL cells | 0.27 | 99.7 |
| Pig LCL cells + Jurkat WT | 88.6 | 11.4 |
| MV (2 hours) | | |
| Pig LCL cells + Jurkat WT | 98 | 1.99 |
| Exosomes (2 hours) | | |
| Pig LCL cells | 98.3 | 1.70 |
| hCD47-Tg LCL cells | 0.098 | 99.9 |
| Pig LCL cells + Jurkat WT | 82.6 | 17.4 |
| MV (6 hours) | | |
| Pig LCL cells + Jurkat WT | 97.8 | 2.20 |
| Exosomes (6 hours) | | |

These data indicate that pig LCL cells (expressing porcine CD47) can be cross-dressed with human CD47 by exposure to EVs or exosomes isolated from wildtype human Jurkat cells.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A method of xenotransplantation, the method comprising
a. obtaining an organ from a donor swine;
b. cross-dressing the organ with human CD47; and
c. transplanting the organ into a human recipient.

2. The method of claim 1, wherein the cross-dressing step comprises exposing the organ to human CD47 comprising extracellular vesicles (EVs).

3. The method of claim 2, wherein the EVs are isolated from human cells.

4. The method of claim 3, wherein the cells express recombinant human CD47.

5. The method of claim 3 or 4, wherein the cells are transgenic cells.

6. The method of any one of claims 2 to 5, wherein the cross-dressing is achieved by incubating the organ with EVs expressing human CD47 for 2 hours.

7. The method of any one of claims 2 to 5, wherein the cross-dressing is achieved by incubating the organ with EVs expressing human CD47 for 6 hours.

8. The method of any one of claims 2 to 5, wherein the cross-dressing is achieved by ex vivo perfusing the organ.

9. The method of any one of claims 2 to 5, wherein the cross-dressing is achieved by in vivo perfusing the donor swine, the human recipient, or a combination thereof.

10. The method of any one of claims 1 to 9, wherein the method results in decreased phagocytosis by human macrophages.

11. The method of any one of claims 1 to 9, wherein the method results in decreased phagocytosis of the cross-dressed organ cells by human macrophages by about 5% to about 25% compared to non-cross-dressed organ cells as measured by FACS analysis of the percentage of CD14-positive cells engulfing cross-dressed cells.

12. The method of any one of claims 1 to 9, wherein the method results in decreased phagocytosis of the cross-dressed organ cells by human macrophages by about 25% to about 50% compared to a non-cross-dressed organ cells as measured by FACS analysis of the percentage of CD14-positive cells engulfing cross-dressed cells.

13. The method of any one of claims 1 to 9, wherein the method results in decreased phagocytosis of the cross-dressed organ cells by human macrophages by about 50% to about 75% compared to a non-cross-dressed organ cell as measured by FACS analysis of the percentage of CD14-positive cells engulfing cross-dressed cells.

14. The method of any one of claims 1 to 9, wherein the method results in decreased phagocytosis of the cross-dressed organ cells by human macrophages by about 75% to about 80% compared to a non-cross-dressed organ cells as measured by FACS analysis of the percentage of CD14-positive cells engulfing cross-dressed cells.

15. The method of any one of claims 1 to 9, wherein the method results in decreased phagocytosis of the cross-dressed organ cells by human macrophages by about 80% to about 85% compared to a non-cross-dressed organ cells as measured by FACS analysis of the percentage of CD14-positive cells engulfing cross-dressed cells.

16. The method of any one of claims 1 to 9, wherein the method results in decreased phagocytosis of the cross-dressed organ cells by human macrophages by about 85% to about 90% compared to a non-cross-dressed organ cells as measured by FACS analysis of the percentage of CD14-positive cells engulfing cross-dressed cells.

17. The method of any one of claims 1 to 9, wherein the method results in decreased phagocytosis of the cross-dressed organ cells by human macrophages by about 90% to about 95% compared to a non-cross-dressed organ cells as measured by FACS analysis of the percentage of CD14-positive cells engulfing cross-dressed cells.

18. The method of any one of claims 1 to 9, wherein the method results in no detectable phagocytosis of the cross-dressed organ as measured by FACS analysis of the percentage of CD14-positive cells engulfing cross-dressed cells.

19. The method of any one of claims 1 to 9, wherein the method results in increased viability of the organ by protection from human macrophages as measured by FACS analysis of the percentage of CD14-positive cells engulfing cross-dressed cells.

20. The method of any one of claims 1 to 19, wherein the cross-dressed organ evades phagocytosis without induction of apoptosis.

21. The method of any one of claims 1 to 19, wherein the cross-dressed organ evades phagocytosis and does not exhibit any detectable level of apoptosis.

22. The method of any one of claims 1 to 19, wherein cells obtained from the cross-dressed organ exhibit about 5% to about 25% lower levels of apoptosis compared to cells obtained from a non-cross-dressed organ.

23. The method of any one of claims 1 to 19, wherein cells obtained from the cross-dressed organ exhibit about 25% to about 50% lower levels of apoptosis compared to cells obtained from a non-cross-dressed organ.

24. The method of any one of claims 1 to 19, wherein cells obtained from the cross-dressed organ exhibit about 50% to about 75% lower levels of apoptosis compared to cells obtained from a non-cross-dressed organ.

25. The method of any one of claims 1 to 19, wherein cells obtained from the cross-dressed organ exhibit about 75% to about 80% lower levels of apoptosis compared to cells obtained from a non-cross-dressed organ.

26. The method of any one of claims 1 to 19, wherein cells obtained from the cross-dressed organ exhibit about 80% to about 85% lower levels of apoptosis compared to cells obtained from a non-cross-dressed organ.

27. The method of any one of claims 1 to 19, wherein cells obtained from the cross-dressed organ exhibit about 85% to about 90% lower levels of apoptosis compared to cells obtained from a non-cross-dressed organ.

28. The method of any one of claims 1 to 19, wherein cells obtained from the cross-dressed organ exhibit at least 90% lower levels of apoptosis compared to cells obtained from a non-cross-dressed organ.

29. The method of any one of claims 20 to 28, wherein apoptosis is measured by propidium iodine staining.

30. The method of any one of claims 1 to 29, wherein the cross-dressed organ exhibits reduced inflammation, compared to a non-cross-dressed organ.

31. The method of any one of claims 1 to 29, wherein the cross-dressed organ exhibits about 5% to about 25% reduced inflammation, compared to a non-cross-dressed organ.

32. The method of any one of claims 1 to 29, wherein the cross-dressed organ exhibits about 25% to about 50% reduced inflammation, compared to a non-cross-dressed organ.

33. The method of any one of claims 1 to 29, wherein the cross-dressed organ exhibits about 50% to about 75% reduced inflammation, compared to a non-cross-dressed organ.

34. The method of any one of claims 1 to 29, wherein the cross-dressed organ exhibits about 75% to about 80% reduced inflammation, compared to a non-cross-dressed organ.

35. The method of any one of claims 1 to 29, wherein the cross-dressed organ exhibits about 80% to about 85% reduced inflammation, compared to a non-cross-dressed organ.

36. The method of any one of claims 1 to 29, wherein the cross-dressed organ exhibits about 85% to about 90% reduced inflammation, compared to a non-cross-dressed organ.

37. The method of any one of claims 1 to 29, wherein the cross-dressed organ exhibits at least 90% reduced inflammation, compared to a non-cross-dressed organ.

38. The method of any one of claims 1 to 37, wherein the human recipient exhibits reduced systemic inflammation post transplantation, compared to transplantation with a non-cross-dressed organ.

39. The method of any one of claims 1 to 37, wherein the human recipient exhibits about 5% to about 25% reduced systemic inflammation post transplantation, compared to transplantation with a non-cross-dressed organ.

40. The method of any one of claims 1 to 37, wherein the human recipient exhibits about 25% to about 50% reduced systemic inflammation post transplantation, compared to transplantation with a non-cross-dressed organ.

41. The method of any one of claims 1 to 37, wherein the human recipient exhibits about 50% to about 75% reduced systemic inflammation post transplantation, compared to transplantation with a non-cross-dressed organ.

42. The method of any one of claims 1 to 37, wherein the human recipient exhibits about 75% to about 80% reduced systemic inflammation post transplantation, compared to transplantation with a non-cross-dressed organ.

43. The method of any one of claims 1 to 37, wherein the human recipient exhibits about 80% to about 85% reduced systemic inflammation post transplantation, compared to transplantation with a non-cross-dressed organ.

44. The method of any one of claims 1 to 37, wherein the human recipient exhibits about 85% to about 90% reduced systemic inflammation post transplantation, compared to transplantation with a non-cross-dressed organ.

45. The method of any one of claims 1 to 37, wherein the human recipient exhibits at least 90% reduced systemic inflammation post transplantation, compared to transplantation with a non-cross-dressed organ.

46. The method of any one of claims 1 to 45, wherein the organ is a kidney.

47. The method of any one of claims 1 to 45, wherein the organ is a lung.

48. The method of claim 46, wherein the human recipient suffers from renal failure.

49. The method of any one of claims 1 to 48, wherein the human recipient requires less immunosuppressive therapy than the standard of care in a comparable clinical setting.

50. The method of any one of claims 1 to 48, wherein the human recipient requires 10-20% less immunosuppressive therapy than the standard of care in a comparable clinical setting.

51. The method of any one of claims 1 to 48, wherein the human recipient requires 20-30% less immunosuppressive therapy than the standard of care in a comparable clinical setting.

52. The method of any one of claims 1 to 48, wherein the human recipient requires 30-40% less immunosuppressive therapy than the standard of care in a comparable clinical setting.

53. The method of any one of claims 1 to 48, wherein the human recipient requires 40-50% less immunosuppressive therapy than the standard of care in a comparable clinical setting.

54. The method of any one of claims 1 to 48, wherein the human recipient requires 50-60% less immunosuppressive therapy than the standard of care in a comparable clinical setting.

55. The method of any one of claims 1 to 48, wherein the human recipient requires 60-70% less immunosuppressive therapy than the standard of care in a comparable clinical setting.

56. The method of any one of claims 1 to 48, wherein the human recipient requires 70-80% less immunosuppressive therapy than the standard of care in a comparable clinical setting.

57. The method of any one of claims 1 to 48, wherein the human recipient requires 80-90% less immunosuppressive therapy than the standard of care in a comparable clinical setting.

58. The method of any one of claims 1 to 48, wherein the human recipient requires at least 90% less immunosuppressive therapy than the standard of care in a comparable clinical setting.

59. The method of any one of claims 1 to 48, wherein the method results in a reduction of proteinuria.

60. The method of any one of claims 1 to 48, wherein the proteinuria is reduced to less than 3 g per 24 hours.

61. The method of any one of claims 1 to 48, wherein the proteinuria is reduced to 500 mg per 24 hours.

62. The method of any one of claims 1 to 48, wherein the proteinuria is reduced to 300 mg per 24 hours.

63. The method of any one of claims 1 to 48, wherein the proteinuria is reduced to 150 mg per 24 hours.

64. The method of any one of claims 1 to 48, wherein the proteinuria resolves within two weeks of the transplant.

65. The method of any one of claims 1 to 48, wherein the proteinuria resolves within one month of the transplant.

66. The method of any one of claims 1 to 48, wherein the proteinuria resolves within two months of the transplant.

67. The method of any one of claims 1 to 48, wherein the proteinuria resolves within four months of the transplant.

68. The method of any one of claims 1 to 67, further comprising transplanting bone marrow tissue into the recipient.

69. The method of claim 68, wherein the bone marrow is taken from the same swine as the kidney.

70. The method of claim 68, wherein the bone marrow is taken from a different swine than the kidney.

71. The method of claim 68, wherein the bone marrow is cross-dressed with human CD47 by exposure to EVs.

72. The method of any one of claims 1 to 71, wherein the organ does not express human SIRPα.


SEQUENCE LISTING (9 sequences)

SEQ ID NO: 1 - protein, 323 aa, Homo sapiens
  source 1..323: note = leukocyte surface antigen CD47 isoform 1 precursor
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 305 aa, Homo sapiens
  source 1..305: note = CD47 molecule
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 3 - protein, 312 aa, Homo sapiens
  source 1..312: note = leukocyte surface antigen CD47 isoform 3 precursor
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 4 - protein, 293 aa, Homo sapiens
  source 1..293: note = leukocyte surface antigen CD47 isoform X2
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 5 - RNA, 5292 nt, Homo sapiens
  source 1..5292: mol_type = mRNA; note = CD47 molecule (CD47), transcript variant 1, mRNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 6 - RNA, 5288 nt, Homo sapiens
  source 1..5288: mol_type = mRNA; note = CD47 molecule (CD47), transcript variant 2, mRNA
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 7 - RNA, 5008 nt, Homo sapiens
  source 1..5008: mol_type = mRNA; note = CD47 molecule (CD47), transcript variant X11
gtgcgcgcggccgtgcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagcta

SEQ ID NO: 8 - RNA, 5259 nt, Homo sapiens
  source 1..5259: mol_type = mRNA; note = CD47 molecule (CD47), transcript variant 3
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 9 - protein, 324 aa, Mus musculus
  source 1..324: note = leukocyte surface antigen CD47 isoform 4 precursor
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR